(19) United States
(12) Patent Application Publication
(10) Pub. No.: US 2011/0257168 A1
(43) Pub. Date: Oct. 20, 2011

(54) DERIVATIVES OF OXABISPIDINE AS NEURONAL NICOTINIC ACETYLCHOLINE RECEPTOR LIGANDS

(75) Inventors: Srinivasa R. Akireddy, Winston-Salem, NC (US); Scott R. Breining, Winston-Salem, NC (US); Nikolai Fedorov, Winston-Salem, NC (US); David Kombo, Winston-Salem, NC (US); Anatoly A. Mazurov, Greensboro, NC (US); Lan Miao, Advance, NC (US); Srinivasa V. Murthy, Lewisville, NC (US); Todd Showalter, Clemmons, NC (US); Yunde Xiao, Clemmons, NC (US); Daniel Yohannes, Winston-Salem, NC (US)

(73) Assignee: Targacept, Inc., Winston-Salem, NC (US)

(21) Appl. No.: 13/055,085

(22) PCT Filed: Jul. 1, 2009
(86) PCT No.: PCT/US2009/049373

§ 371 Date: Jul. 6, 2011

Related U.S. Application Data
(60) Provisional application No. 61/078,005, filed on Jul. 3, 2008.

(51) Int. Cl. A61K 31/5383; A61P 29/00; C07D 498/04

(52) U.S. Cl. 514/230.5; 544/74

(57) ABSTRACT

The present invention relates to compounds of formula (I) that bind to and modulate the activity of neuronal nicotinic acetylcholine receptors, to processes for preparing these compounds, to pharmaceutical compositions containing these compounds, and to methods of using these compounds for treating a wide variety of conditions and disorders, including those associated with dysfunction of the central nervous system (CNS).

### FIELD OF THE INVENTION

The present invention relates to compounds that bind to and modulate the activity of neuronal nicotinic acetylcholine receptors, to processes for preparing these compounds, to pharmaceutical compositions containing these compounds, and to methods of using these compounds for treating a wide variety of conditions and disorders, including those associated with dysfunction of the central nervous system (CNS).

### BACKGROUND OF THE INVENTION

The therapeutic potential of compounds that target neuronal nicotinic receptors (NNRs), also known as nicotinic acetylcholine receptors (nAChRs), has been the subject of several reviews. See, for example, Breining et al., Ann. Rep. Med. Chem. 40: 3 (2005), Hogg and Bertrand, Curr. Drug Targets: CNS Neurol. Disord. 3: 123 (2004), Suto and Zacharias, Expert Opin. Ther. Targets 8: 61 (2004), Dani et al., Bioorg. Med. Chem. Lett. 14: 1837 (2004), Bencherif and Schmitt, Curr. Drug Targets: CNS Neurol. Disord. 1: 349 (2002), each incorporated by reference with regard to such teaching. Among the kinds of indications for which NNR ligands have been proposed as therapies are cognitive disorders, including Alzheimer's disease, attention deficit disorder, and schizophrenia (Newhouse et al., Curr. Opin. Pharmacol. 4: 36 (2004), Levin and Rezvani, Curr. Drug Targets: CNS Neurol. Disord. 1: 423 (2002), Graham et al., Curr. Drug Targets: CNS Neurol. Disord. 1: 387 (2002), Ripoll et al., Curr. Med. Res. Opin. 20(7): 1057 (2004), and McEvoy and Allen, Curr. Drug Targets: CNS Neurol. Disord. 1: 433 (2002)); pain and inflammation (Decker et al., Curr. Top. Med. Chem. 4(3): 369 (2004), Vincler, Expert Opin. Invest. Drugs 14(10): 1191 (2005), Jain, Curr. Opin. Inv. Drugs 5: 76 (2004), Miao et al., Neuroscience 123: 777 (2004)); depression and anxiety (Shytle et al., Mol. Psychiatry 7: 525 (2002), Damaj et al., Mol. Pharmacol. 66: 675 (2004), Shytle et al., Depress. Anxiety 16: 89 (2002)); neurodegeneration (O'Neill et al., Curr. Drug Targets: CNS Neurol. Disord. 1: 399 (2002), Takata et al., J. Pharmacol. Exp. Ther. 306: 772 (2003), Marrero et al., J. Pharmacol. Exp. Ther. 309: 16 (2004)); Parkinson's disease (Jonnala and Buccafusco, J. Neurosci. Res. 66: 565 (2001)); addiction (Dwoskin and Crooks, Biochem. Pharmacol. 63: 89 (2002), Coe et al., Bioorg. Med. Chem. Lett. 15(22): 4889 (2005)); obesity (Li et al., Curr. Top. Med. Chem. 3: 899 (2003)); and Tourette's syndrome (Sacco et al., J. Psychopharmacol. 18(4): 457 (2004), Young et al., Clin. Ther. 23(4): 532 (2001)), each of these references incorporated by reference with regard to the nexus of the receptor and the named indication(s).

A limitation of some nicotinic compounds is that they are associated with various undesirable side effects which can occur, for example, by stimulating muscle and ganglionic receptors. Therefore, there is a need to have compounds, compositions, and methods for preventing or treating various conditions or disorders where the compounds exhibit a high enough degree of nAChR subtype specificity to elicit a beneficial effect, without significantly affecting those receptor subtypes which have the potential to induce undesirable side effects, including, for example, appreciable activity at cardiovascular and skeletal muscle sites.

### SUMMARY OF THE INVENTION

The present invention includes compounds of Formula I:

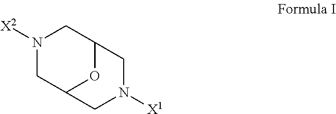

wherein:

X¹ is aryl (optionally substituted with one or more R groups) or heteroaryl (optionally substituted with one or more R groups);

each R independently is C₁₋₆ alkyl, C₂₋₆ alkenyl, C₂₋₆ alkynyl, C₃₋₈ cycloalkyl, —(CH₂)_{q}C₃₋₈cycloalkyl, heterocyclyl, —(CH₂)_{q}heterocyclyl, aryl, —(CH₂)_{q}aryl, heteroaryl, —(CH₂)_{q}heteroaryl, halo, —OR^{I}, —NR^{I}R^{II}, C₁₋₆ haloalkyl , —CN, —NO₂, —C₂R^{I}, —SR^{I}, —N₃, —C(═O)NR^{I}R^{II}, —NR^{I}C(═O)R^{II}, —OC(═O)NR^{I}R^{II}, —NR^{I}C(═O)OR^{II}, —SO₂R^{I}, —SO₂NR^{I}R^{II}, or —NR^{I}SO₂R^{II};

each of R^{I} and R^{II} independently is hydrogen, C₁₋₆ alkyl, C₁₋₆ haloalkyl, C₃₋₈ cycloalkyl, —(CH₂)_{q}C₃₋₈ cycloalkyl, heterocyclyl, —(CH₂)_{q}heterocyclyl, aryl (optionally substituted with one or more C₁₋₆ alkyl, halogen, or C₁₋₆ haloalkyl), —(CH₂)_{q}aryl (optionally substituted with one or more C₁₋₆ alkyl, halogen, or C₁₋₆ haloalkyl), heteroaryl (optionally substituted with one or more C₁₋₆ alkyl, halogen, or C₁₋₆ haloalkyl), or —(CH₂)_{q}heteroaryl (optionally substituted with one or more C₁₋₆ alkyl, halogen, or C₁₋₆ haloalkyl), or

R^{I} and R^{II} can combine together with the atoms to which they are attached to form a three to ten membered ring;

each q independently is 1, 2, 3, 4, 5, or 6;

X² is hydrogen, C₁₋₆ alkyl, cycloalkyl, —(CH₂)_{q}C₃₋₈cycloalkyl, —(CH₂)_{q}aryl, or —(CH₂)_{q}heteroaryl;

or a pharmaceutically acceptable salt thereof.

The compounds of the present invention bind with high affinity to NNRs of the α4β2 and α7 subtypes, found in the CNS. The present invention also relates to pharmaceutically acceptable salts prepared from these compounds.

The present invention includes pharmaceutical compositions comprising a compound of the present invention or a pharmaceutically acceptable salt thereof. The pharmaceutical compositions of the present invention can be used for treating or preventing a wide variety of conditions or disorders, and particularly those disorders characterized by dysfunction of nicotinic cholinergic neurotransmission or the degeneration of the nicotinic cholinergic neurons.

The present invention includes a method for treating or preventing disorders and dysfunctions, such as CNS disorders and dysfunctions, and also for treating or preventing certain conditions, for example, alleviating pain and inflammation, in mammals in need of such treatment. The methods involve administering to a subject a therapeutically effective amount of a compound of the present invention, including a salt thereof, or a pharmaceutical composition that includes such compounds.

### BRIEF DESCRIPTION OF THE FIGURES

FIG. 1 is a graphic illustration demonstrating the effects of Compound A in significantly reducing nociceptive behavior in a formalin test upon s.c. administration, including illustration of a lowest active dose of 3 mg/kg.

### DETAILED DESCRIPTION OF THE INVENTION

### I. COMPOUNDS

One embodiment of the present invention includes a compound as represented by Formula I:

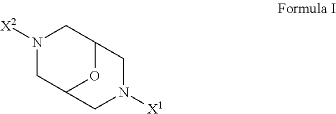

wherein:

X¹ is aryl (optionally substituted with one or more R groups) or heteroaryl (optionally substituted with one or more R groups);

each R independently is C₁₋₆ alkyl, C₂₋₆ alkenyl, C₂₋₆ alkynyl, C₃₋₈ cycloalkyl, —(CH₂)_{q}C₃₋₈cycloalkyl, heterocyclyl, —(CH₂)_{q}heterocyclyl, aryl, —(CH₂)_{q}aryl, heteroaryl, —(CH₂)_{q}heteroaryl, halo, —OR^{I}, —NR^{I}R^{II}, C₁₋₆ haloalkyl, —CN, —NO₂, —C₂R^{I}, —SR^{I}, —N₃, —C(═O)NR^{I}R^{II}, —NR^{I}C(═O)R^{II}, —OC(═O)NR^{I}R^{II}, —NR^{I}C(═O)OR^{II}, —SO₂R^{I}, —SO₂NR^{I}R^{II}, or —NR^{I}SO₂R^{II};

each R^{I} and R^{II} independently is hydrogen, C₁₋₆ alkyl, C₁₋₆ haloalkyl, C₃₋₈ cycloalkyl, —(CH₂)_{q}C₃₋₈cycloalkyl, heterocyclyl, —(CH₂)_{q}heterocyclyl, aryl (optionally substituted with one or more C₁₋₆ alkyl, halogen, or C₁₋₆ haloalkyl), —(CH₂)_{q}aryl (optionally substituted with one or more C₁₋₆ alkyl, halogen, or C₁₋₆ haloalkyl), heteroaryl (optionally substituted with one or more C₁₋₆ alkyl, halogen, or C₁₋₆ haloalkyl), or —(CH₂)_{q}heteroaryl (optionally substituted with one or more C₁₋₆ alkyl, halogen, or C₁₋₆ haloalkyl), or R^{I} and R^{II} can combine together with the atoms to which they are attached to form a three to ten membered ring;

each q independently is 1, 2, 3, 4, 5, or 6;

X² is hydrogen, C₁₋₆ alkyl, cycloalkyl, —(CH₂)_{q}C₃₋₈cycloalkyl, —(CH₂)_{q}aryl, or —(CH₂)_{q}heteroaryl;

or a pharmaceutically acceptable salt thereof.

In one embodiment, X¹ is unsubstituted or substituted pyridine, pyridazine, pyrimidine, phenyl, or pyrazine. In a further embodiment, X¹ is substituted with one or more halogen, C₁₋₆ alkoxy, C₁₋₆ haloalkoxy, —(CH₂)_{q}C₃₋₈cycloalkyl, C₁₋₆ alkyl, —CN, —OR^{I}, —NR^{I}R^{II}, or aryl.

In one embodiment, X² is hydrogen or C₁₋₆ alkyl.

In one embodiment, a compound is selected from:
3-(6-chloropyridazin-3-yl)-9-oxa-3,7-diazabicyclo[3.3.1]nonane;
3-(5-methoxypyridin-3-yl)-9-oxa-3,7-diazabicyclo[3.3.1]nonane;
3-(5-isopropoxypyridin-3-yl)-9-oxa-3,7-diazabicyclo[3.3.1]nonane;
3-(5,6-dichloropyridin-3-yl)-9-oxa-3,7-diazabicyclo[3.3.1]nonane;
3-(5-trifluoromethylpyridin-3-yl)-9-oxa-3,7-diazabicyclo[3.3.1]nonane;
3-(5-methoxy-6-chloropyridin-3-yl)-9-oxa-3,7-diazabicyclo[3.3.1]nonane;
3-(5-(cyclopropylmethoxy)pyridin-3-yl)-9-oxa-3,7-diazabicyclo[3.3.1]nonane;
3-(5-bromopyridin-3-yl)-9-oxa-3,7-diazabicydo[3.3.1]nonane;
3-(pyrimidin-5-yl)-9-oxa-3,7-diazabicyclo[3.3.1]nonane;
3-(pyridin-3-yl)-9-oxa-3,7-diazabicyclo[3.3.1]nonane;
3-(5-(3,4-dichlorophenoxy)pyridin-3-yl)-7-methyl-9-oxa-3,7-diazabicyclo[3.3.1]nonane;
3-(pyridin-4-yl)-7-methyl-9-oxa-3,7-diazabicyclo[3.3.1]nonane;
3-(6-trifluoromethyl-pyridin-3-yl)-7-methyl-9-oxa-3,7-diazabicyclo[3.3.1]nonane;
3-(5-fluoropyridin-3-yl)-7-methyl-9-oxa-3,7-diazabicyclo[3.3.1]nonane;
3-(6-fluoropyridin-3-yl)-7-methyl-9-oxa-3,7-diazabicyclo[3.3.1]nonane;
3-(2,3-difluorophenyl)-7-methyl-9-oxa-3,7-diazabicyclo[3.3.1]nonane;
3-(6-phenylpyridazin-3-yl)-7-methyl-9-oxa-3,7-diazabicyclo[3.3.1]nonane;
3-(4-cyanopyridin-3-yl)-7-methyl-9-oxa-3,7-diazabicyclo[3.3.1]nonane;
3-(pyrimidin-5-yl)-7-methyl-9-oxa-3,7-diazabicyclo[3.3.1]nonane;
3-(6-dimethylaminopyridin-3-yl)-7-methyl-9-oxa-3,7-diazabicyclo[3.3.1]nonane;
3-(3-methoxypyridin-2-yl)-7-methyl-9-oxa-3,7-diazabicyclo[3.3.1]nonane;
3-(5-methylpyridin-3-yl)-7-methyl-9-oxa-3,7-diazabicyclo[3.3.1]nonane;
3-(5-chloropyridin-3-yl)-7-methyl-9-oxa-3,7-diazabicyclo[3.3.1]nonane;
3-(3-methoxyphenyl)-7-methyl-9-oxa-3,7-diazabicyclo[3.3.1]nonane;
3-(3,5-difluorophenyl)-7-methyl-9-oxa-3,7-diazabicyclo[3.3.1]nonane;
3-(5-cyanopyridin-3-yl)-7-methyl-9-oxa-3,7-diazabicyclo[3.3.1]nonane;
3-(6-chloropyridazin-3-yl)-7-methyl-9-oxa-3,7-diazabicyclo[3.3.1]nonane;
3-(6-methoxypyridin-3-yl)-7-methyl-9-oxa-3,7-diazabicyclo[3.3.1]nonane;
3-(6-chloropyridin-3-yl)-7-methyl-9-oxa-3,7-diazabicyclo[3.3.1]nonane;
3-(5-(2-chlorophenoxy)pyridin-3-yl)-7-methyl-9-oxa-3,7-diazabicyclo[3.3.1 ]nonane;
3-(6-methoxypyridin-2-yl)-7-methyl-9-oxa-3,7-diazabicyclo[3.3.1]nonane;
3-(6-methylpyridin-3-yl)-7-methyl-9-oxa-3,7-diazabicyclo[3.3.1]nonane;
3-(6-(furan-3-yl)pyridazin-3-yl)-7-methyl-9-oxa-3,7-diazabicyclo[3.3.1]nonane;
3-(2,3-dichlorophenyl)-7-methyl-9-oxa-3,7-diazabicyclo[3.3.1]nonane;
3-(6-cyanopyridin-3-yl)-7-methyl-9-oxa-3,7-diazabicyclo[3.3.1]nonane;
3-(pyridin-2-yl)-7-methyl-9-oxa-3,7-diazabicyclo[3.3.1]nonane;
3-(3-methoxypyridin-2-yl)-9-oxa-3,7-diazabicyclo[3.3.1]nonane;
3-(2,3-difluorophenyl)-9-oxa-3,7-diazabicyclo[3.3.1 ]nonane;
3-(4-methoxypyridin-3-yl)-9-oxa-3,7-diazabicyclo[3.3.1]nonane;
3-(4-chloropyridin-3-yl)-9-oxa-3,7-diazabicydo[3.3.1]nonane;
3-(6-fluoropyridin-3-yl)-9-oxa-3,7-diazabicyclo[3.3.1]nonane;
3-(6-cyanopyridin-3-yl)-9-oxa-3,7-diazabicyclo[3.3.1]nonane;
3-(5-methylpyridin-3-yl)-9-oxa-3,7-diazabicydo[3.3.1]nonane;
3-(6-dimethylaminopyridin-3-yl)-9-oxa-3,7-diazabicyclo[3.3.1]nonane;
3-(pyrazin-2-yl)-9-oxa-3,7-diazabicyclo[3.3.1]nonane;
3-(pyridin-2-yl)-9-oxa-3,7-diazabicyclo[3.3.1]nonane;
3-(6-methoxypyridin-3-yl)-9-oxa-3,7-diazabicyclo[3.3.1]nonane;
3-(pyridin-4-yl)-9-oxa-3,7-diazabicyclo[3.3.1]nonane;
3-(5-(3,4-dichlorophenoxy)pyridin-3-yl)-9-oxa-3,7-diazabicyclo[3.3.1]nonane;
3-(4-cyanopyridin-3-yl)-9-oxa-3,7-diazabicyclo[3.3.1]nonane;
3-(3,5-difluorophenyl)-9-oxa-3,7-diazabicyclo[3.3.1]nonane;
3-(6-chloropyridin-3-yl)-9-oxa-3,7-diazabicydo[3.3.1]nonane;
3-(6-methoxypyridin-2-yl)-9-oxa-3,7-diazabicyclo[3.3.1]nonane;
3-(2,3-dichlorophenyl)-9-oxa-3,7-diazabicyclo[3.3.1]nonane;
3-(6-methylpyridin-3-yl)-9-oxa-3,7-diazabicydo[3.3.1]nonane;
3-(6-phenylpyridazin-3-yl)-9-oxa-3,7-diazabicyclo[3.3.1]nonane;
3-(5-(2-chlorophenoxy)pyridin-3-yl)-9-oxa-3,7-diazabicyclo[3.3.1]nonane;
3-(5-fluoropyridin-3-yl)-9-oxa-3,7-diazabicyclo[3.3.1]nonane;
3-(3-methoxyphenyl)-9-oxa-3,7-diazabicyclo[3.3.1]nonane;
3-(6-trifluoromethylpyridin-3-yl)-9-oxa-3,7-diazabicyclo[3.3.1]nonane;
3-(5-chloropyridin-3-yl)-9-oxa-3,7-diazabicydo[3.3.1]nonane;
3-(2-phenylpyrimidin-5-yl)-9-oxa-3,7-diazabicyclo[3.3.1]nonane;
3-(6-(furan-3-yl)pyridazin-3-yl)-9-oxa-3,7-diazabicyclo[3.3.1]nonane;
3-(6-(pyridin-3-yl)pyridazin-3-yl)-9-oxa-3,7-diazabicyclo[3.3.1]nonane;
3-(5-cyanopyridin-3-yl)-9-oxa-3,7-diazabicyclo[3.3.1]nonane;
3-(pyrazin-2-yl)-7-methyl-9-oxa-3,7-diazabicyclo[3.3.1]nonane;
3-(2-phenylpyrimidin-5-yl)-7-methyl-9-oxa-3,7-diazabicyclo[3.3.1]nonane;
3-(6-(pyridin-3-yl)pyridazin-3-yl)-7-methyl-9-oxa-3,7-diazabicyclo[3.3.1]nonane;
3-(4-chloropyridin-3-yl)-7-methyl-9-oxa-3,7-diazabicyclo[3.3.1]nonane;
3-(2-bromopyrimidin-5-yl)-9-oxa-3,7-diazabicyclo[3.3.1 ]nonane;
3-(4-methoxypyridin-3-yl)-7-methyl-9-oxa-3,7-diazabicyclo[3.3.1 ]nonane;
3-(2-bromopyrimidin-5-yl)-7-methyl-9-oxa-3,7-diazabicyclo[3.3.1]nonane;
3-(furo[3,2-b]pyridin-6-yl)-9-oxa-3,7-diazabicyclo[3.3.1]nonane;
3-(5-fluoro-pyridine-1-oxide-3-yl)-9-oxa-3,7-diazabicyclo[3.3.1]nonane;
3-methyl-7-(pyridin-3-yl)-9-oxa-3,7-diazabicyclo[3.3.1]nonane;
3-methyl-7-(5,6-dichloropyridin-3-yl)-9-oxa-3,7-diazabicyclo[3.3.1]nonane;
3-(furo[2,3-b]pyridin-5-yl)-9-oxa-3,7-diazabicyclo[3.3.1]nonane;
3-(5-(difluoromethoxy)pyridin-3-yl)-9-oxa-3,7-diazabicyclo[3.3.1]nonane;
3-(2,2-difluorobenzo[d][1,3]dioxol-5-yl)-9-oxa-3,7-diazabicyclo[3.3.1]nonane;
3-(6-chloro-5-(difluoromethoxy)pyridin-3-yl)-9-oxa-3,7-diazabicyclo[3.3.1]nonane;
3-(6-cyano-5-(difluoromethoxy)pyridin-3-yl)-9-oxa-3,7-diazabicyclo[3.3.1]nonane;
3-(5-cyano-6-fluoropyridin-3-yl)-9-oxa-3,7-diazabicyclo[3.3.1]nonane;
3-(5-methoxy-6-fluoropyridin-3-yl)-9-oxa-3,7-diazabicyclo[3.3.1]nonane;
3-(5-cyclopropylpyridin-3-yl)-9-oxa-3,7-diazabicyclo[3.3.1]nonane;
3-(2,2-difluoro-[1,3]dioxolo[4,5-b]pyridine-5-yl)-9-oxa-3,7-diazabicyclo[3.3.1]nonane;
3-(5-cyclopropyl-6-chloropyridin-3-yl)-9-oxa-3,7-diazabicyclo[3.3.1]nonane;
3-(5-(3-fluoropropoxy)-6-chloropyridin-3-yl)-9-oxa-3,7-diazabicyclo[3.3.1]nonane;
3-(5-(4-fluorobutoxy)-6-chloropyridin-3-yl)-9-oxa-3,7-diazabicyclo[3.3.1]nonane;
and
3-(5-(2-fluoroethoxy)-6-chloropyridin-3-yl)-9-oxa-3,7-diazabicyclo[3.3.1]nonane;
or a pharmaceutically acceptable salt thereof.

One aspect of the present invention includes a compound:

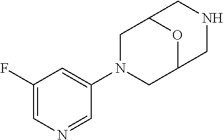

or a pharmaceutically acceptable salt thereof. Within this specification, this compound may be referred to by chemical name, which according to differing naming conventions may be 3-(5-fluoropyridin-3-yl)-9-oxa-3,7-diazabicyclo[3.3.1]nonane or 7-(5-fluoro-3-pyridyl)-9-oxa-3,7-diazabicyclo[3.3.1]nonane, or may also be referred to as Compound A.

One aspect of the present invention includes a pharmaceutical composition comprising a compound of the present invention and a pharmaceutically acceptable carrier.

One aspect of the present invention includes a method for the treatment or prevention of a disease or condition mediated by a neuronal nicotinic receptor comprising the administration of a compound of the present invention. In one embodiment, the neuronal nicotinic receptor is of the α4β2 or α7 subtype. In one embodiment, the disease or condition is a CNS disorder. In another embodiment, the disease or condition is inflammation or an inflammatory response associated with one or more of a bacterial or viral infection. In another embodiment, the disease or condition is pain. In another embodiment, the disease or condition is neovascularization. In another embodiment, the disease or condition is another disorder described herein.

One aspect of the present invention includes use of a compound of the present invention for the preparation of a medicament for the treatment or prevention of a disease or condition mediated by a neuronal nicotinic receptor. In one embodiment, the neuronal nicotinic receptor is of the α4β2 or α7 subtype. In one embodiment, the disease or condition is a CNS disorder. In another embodiment, the disease or condition is inflammation or an inflammatory response associated with one or more of a bacterial or viral infection. In another embodiment, the disease or condition is pain. In another embodiment, the disease or condition is neovascularization. In another embodiment, the disease or condition is another disorder described herein.

One aspect of the present invention includes a compound of the present invention for use as an active therapeutic substance. One aspect, thus, includes a compound of the present invention for use in the treatment or prevention of a disease or condition mediated by a neuronal nicotinic receptor. In one embodiment, the neuronal nicotinic receptor is of the α4β2 or α7 subtype. In one embodiment, the disease or condition is a CNS disorder. In another embodiment, the disease or condition is inflammation or an inflammatory response associated with one or more of a bacterial or viral infection. In another embodiment, the disease or condition is pain. In another embodiment, the disease or condition is neovascularization. In another embodiment, the disease or condition is another disorder described herein.

The scope of the present invention includes all combinations of aspects and embodiments.

The following definitions are meant to clarify, but not limit, the terms defined. If a particular term used herein is not specifically defined, such term should not be considered indefinite. Rather, terms are used within their accepted meanings.

As used throughout this specification, the preferred number of atoms, such as carbon atoms, will be represented by, for example, the phrase “C_{x-y} alkyl,” which refers to an alkyl group, as herein defined, containing the specified number of carbon atoms. Similar terminology will apply for other preferred terms and ranges as well. Thus, for example, C₁₋₆ alkyl represents a straight or branched chain hydrocarbon containing one to six carbon atoms.

As used herein the term “alkyl” refers to a straight or branched chain hydrocarbon, which may be optionally substituted, with multiple degrees of substitution being allowed. Examples of “alkyl” as used herein include, but are not limited to, methyl, ethyl, propyl, isopropyl, isobutyl, n-butyl, tert-butyl, isopentyl, and n-pentyl.

As used herein the term “alkenyl” refers to a straight or branched chain aliphatic hydrocarbon containing one or more carbon-to-carbon double bonds, which may be optionally substituted, with multiple degrees of substitution being allowed. Examples of “alkenyl” as used herein include, but are not limited to, vinyl, and allyl.

As used herein the term “alkynyl” refers to a straight or branched chain aliphatic hydrocarbon containing one or more carbon-to-carbon triple bonds, which may be optionally substituted, with multiple degrees of substitution being allowed. An example of “alkynyl” as used herein includes, but is not limited to, ethynyl.

As used herein, the term “cycloalkyl” refers to a fully saturated optionally substituted monocyclic, bicyclic, or bridged hydrocarbon ring, with multiple degrees of substitution being allowed. Exemplary “cycloalkyl” groups as used herein include, but are not limited to, cyclopropyl, cyclobutyl, cyclopentyl, cyclohexyl, and cycloheptyl.

As used herein, the term “heterocycle” or “heterocyclyl” refers to an optionally substituted mono- or polycyclic ring system, optionally containing one or more degrees of unsaturation, and also containing one or more heteroatoms, which may be optionally substituted, with multiple degrees of substitution being allowed. Exemplary heteroatoms include nitrogen, oxygen, or sulfur atoms, including N-oxides, sulfur oxides, and dioxides. Preferably, the ring is three to twelve-membered, preferably three- to eight-membered and is either fully saturated or has one or more degrees of unsaturation. Such rings may be optionally fused to one or more of another heterocyclic ring(s) or cycloalkyl ring(s). Examples of “heterocyclic” groups as used herein include, but are not limited to, tetrahydrofuran, pyran, tetrahydropyran, 1,4-dioxane, 1,3-dioxane, piperidine, pyrrolidine, morpholine, tetrahydrothiopyran, and tetrahydrothiophene.

As used herein, the term “aryl” refers to a single benzene ring or fused benzene ring system which may be optionally substituted, with multiple degrees of substitution being allowed. Examples of “aryl” groups as used include, but are not limited to, phenyl, 2-naphthyl, 1-naphthyl, anthracene, and phenanthrene. Preferable aryl rings have five- to ten-members.

As used herein, a fused benzene ring system encompassed within the term “aryl” includes fused polycyclic hydrocarbons, namely where a cyclic hydrocarbon with less than maximum number of noncumulative double bonds, for example where a saturated hydrocarbon ring (cycloalkyl, such as a cyclopentyl ring) is fused with an aromatic ring (aryl, such as a benzene ring) to form, for example, groups such as indanyl and acenaphthalenyl, and also includes such groups as, for non-limiting examples, dihydronaphthalene and tetrahydronaphthalene.

As used herein, the term “heteroaryl” refers to a monocyclic five to seven membered aromatic ring, or to a fused bicyclic aromatic ring system comprising two of such aromatic rings, which may be optionally substituted, with multiple degrees of substitution being allowed. Preferably, such rings contain five- to ten-members. These heteroaryl rings contain one or more nitrogen, sulfur, and/or oxygen atoms, where N-oxides, sulfur oxides, and dioxides are permissible heteroatom substitutions. Examples of “heteroaryl” groups as used herein include, but are not limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzoxazole, benzothiophene, indole, indazole, benzimidazole, imidazopyridine, pyrazolopyridine, and pyrazolopyrimidine.

As used herein the term “halogen” refers to fluorine, chlorine, bromine, or iodine.

As used herein the term “haloalkyl” refers to an alkyl group, as defined herein, that is substituted with at least one halogen. Examples of branched or straight chained “haloalkyl” groups as used herein include, but are not limited to, methyl, ethyl, propyl, isopropyl, n-butyl, and t-butyl substituted independently with one or more halogens, for example, fluoro, chloro, bromo, and iodo. The term “haloalkyl” should be interpreted to include such substituents as perfluoroalkyl groups such as —CF₃.

As used herein the term “alkoxy” refers to a group —OR^{a}, where R^{a} is alkyl or cycloalkyl as defined above.

As used herein the term “nitro” refers to a group —NO₂.

As used herein the term “cyano” refers to a group —CN.

As used herein the term “azido” refers to a group —N₃.

As used herein “amino” refers to a group —NR^{a}R^{b}, where each of R^{a} and R^{b} individually is hydrogen, alkyl, alkenyl, alkynyl, cycloalkyl, aryl, heterocylcyl, or heteroaryl. As used herein, when either R^{a} or R^{b} is other than hydrogen, such a group may be referred to as a “substituted amino” or, for example if R^{a} is H and R^{b} is alkyl, as an “alkylamino.”

As used herein, the term “hydroxyl” refers to a group —OH.

The compounds of this invention may be made by a variety of methods, including well-known standard synthetic methods. Illustrative general synthetic methods are set out below and then specific compounds of the invention are prepared in the working Examples.

In all of the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1999) Protecting Groups in Organic Synthesis, 3^{rd} Edition, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of the present invention.

The present invention also provides a method for the synthesis of compounds useful as intermediates in the preparation of compounds of the present invention along with methods for their preparation.

The compounds can be prepared according to the methods described below using readily available starting materials and reagents. In these reactions, variants may be employed which are themselves known to those of ordinary skill in this art, but are not mentioned in greater detail.

Unless otherwise stated, structures depicted herein are also meant to include compounds which differ only in the presence of one or more isotopically enriched atoms. Compounds having the present structure except for the replacement of a hydrogen atom by a deuterium or tritium, or the replacement of a carbon atom by a ¹³C- or ¹⁴C-enriched carbon are within the scope of the invention. For example, deuterium has been widely used to examine the pharmacokinetics and metabolism of biologically active compounds. Although deuterium behaves similarly to hydrogen from a chemical perspective, there are significant differences in bond energies and bond lengths between a deuterium-carbon bond and a hydrogen-carbon bond. Consequently, replacement of hydrogen by deuterium in a biologically active compound may result in a compound that generally retains its biochemical potency and selectivity but manifests significantly different absorption, distribution, metabolism, and/or excretion (ADME) properties compared to its isotope-free counterpart. Thus, deuterium substitution may result in improved drug efficacy, safety, and/or tolerability for some biologically active compounds.

The compounds of the present invention may crystallize in more than one form, a characteristic known as polymorphism, and such polymorphic forms (“polymorphs”) are within the scope of the present invention. Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point.

Certain of the compounds described herein contain one or more chiral centers, or may otherwise be capable of existing as multiple stereoisomers. The scope of the present invention includes mixtures of stereoisomers as well as purified enantiomers or enantiomerically/diastereomerically enriched mixtures. Also included within the scope of the invention are the individual isomers of the compounds represented by the formulae of the present invention, as well as any wholly or partially equilibrated mixtures thereof. The present invention also includes the individual isomers of the compounds represented by the formulas above as mixtures with isomers thereof in which one or more chiral centers are inverted.

When a compound is desired as a single enantiomer, such may be obtained by stereospecific synthesis, by resolution of the final product or any convenient intermediate, or by chiral chromatographic methods as are known in the art. Resolution of the final product, an intermediate, or a starting material may be effected by any suitable method known in the art. See, for example, Stereochemistry of Organic Compounds (Wiley-Interscience, 1994), incorporated by reference with regard to stereochemistry.

The present invention includes a salt or solvate of the compounds herein described, including combinations thereof such as a solvate of a salt. The compounds of the present invention may exist in solvated, for example hydrated, as well as unsolvated forms, and the present invention encompasses all such forms.

Typically, but not absolutely, the salts of the present invention are pharmaceutically acceptable salts. Salts encompassed within the term “pharmaceutically acceptable salts” refer to non-toxic salts of the compounds of this invention.

Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt, and N,N′-dibenzylethylenediamine salt; and salts with basic amino acid such as lysine salt and arginine salt. The salts may be in some cases hydrates or ethanol solvates. Representative salts are provided as described in U.S. Pat. No. 5,597,919 to Dull et al., U.S. Pat. No. 5,616,716 to Dull et al. and U.S. Pat. No. 5,663,356 to Ruecroft et al, each of which is herein incorporated by reference with regard to such salts.

### II. GENERAL SYNTHETIC METHODS

For compounds of the present invention, the 9-oxa-3,7-diazabicyclo[3.3.1]nonane scaffold is prepared through a modification of the procedure of Stetter et al. (Chem. Ber. 96(11): 2827 (1963), herein incorporated by reference with regard to such synthetic teaching) as illustrated in Scheme 1. Diallylamine 1 is allowed to react with benzylchloroformate in triethylamine to give benzyloxycarbonyl (Cbz) protected diallylamine 2. Reaction of this compound with aqueous mercury(II) acetate yields compound 3, which is subsequently allowed to react with iodine to give diiodo compound 4. Treatment of this compound with methanolic ammonia yields Cbz protected 9-oxa-3,7-diazabicyclo[3.3.1]nonane 5. Protection of the second amine group with t-butoxycarbonyl (Boc) protecting group by reaction of 5 with di-t-butyl dicarbonate (resulting in compound 6) and subsequent removal of the Cbz moiety by hydrogenation over palladium hydroxide yields the Boc-protected 9-oxa-3,7-diazabicyclo[3.3.1]nonane (Boc-oxabispidine) 7.

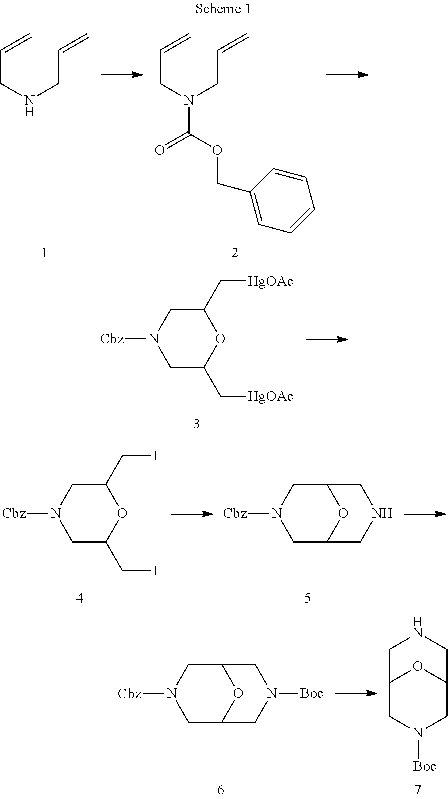

The compounds of the present invention can be prepared via the coupling (often palladium catalyzed) of diazabicycle 7 with a suitably functionalized aryl or heteroaryl halide or other reactive aryl or heteroaryl derivative. Such compounds may be available commercially or may be prepared by a variety of synthetic procedures well known to those of skill in the art of organic synthesis. After N-arylation, removal of the Boc-protecting group (from the other nitrogen) with acid under either aqueous or anhydrous conditions, will afford the compounds of the present invention. Other compounds of the present invention can be synthesized by alkylation of the remaining basic nitrogen with an activated alkyl compound such as an alkyl halide. Other alkylation reactions can also be used. Thus, reaction of a secondary amine with formaldehyde in formic acid results in methylation to give a tertiary amine.

Those skilled in the art of organic synthesis will appreciate that there exist multiple means of producing compounds of the present invention which are labeled with a radioisotope appropriate to various uses. Thus, coupling of a ¹¹C- or ¹⁸F-labeled aryl or heteroaryl halide with either compound 5 or compound 7 followed by removal of the protecting group as described above will produce a compound suitable for use in positron emission tomography. Likewise, coupling of a ³H- or ¹⁴C-labeled aryl or heteroaryl halide with either compound 5 or compound 7 followed by removal of the protecting group as described above will produce a compound suitable for use in receptor binding and metabolism studies.

### III. PHARMACEUTICAL COMPOSITIONS

The pharmaceutical compositions of the present invention include the salts described herein, in the pure state or in the form of a composition in which the compounds are combined with any other pharmaceutically compatible product, which can be inert or physiologically active. The resulting pharmaceutical compositions can be used to prevent a condition or disorder in a subject susceptible to such a condition or disorder, and/or to treat a subject suffering from the condition or disorder. The pharmaceutical compositions described herein include one or more compounds of Formula I and/or pharmaceutically acceptable salts thereof.

The manner in which the compounds are administered can vary. The compositions are preferably administered orally (e.g., in liquid form within a solvent such as an aqueous or non-aqueous liquid, or within a solid carrier). Preferred compositions for oral administration include pills, tablets, capsules, caplets, syrups, and solutions, including hard gelatin capsules and time-release capsules. Standard excipients include binders, fillers, colorants, solubilizers and the like. Compositions can be formulated in unit dose form, or in multiple or subunit doses. Preferred compositions are in liquid or semisolid form. Compositions including a liquid pharmaceutically inert carrier such as water or other pharmaceutically compatible liquids or semisolids can be used. The use of such liquids and semisolids is well known to those of skill in the art.

The compositions can also be administered via injection, i.e., intravenously, intramuscularly, subcutaneously, intraperitoneally, intraarterially, intrathecally; and intracerebroventricularly. Intravenous administration is the preferred method of injection. Suitable carriers for injection are well known to those of skill in the art and include 5% dextrose solutions, saline, and phosphate-buffered saline. The compounds can also be administered as an infusion or injection (e.g., as a suspension or as an emulsion in a pharmaceutically acceptable liquid or mixture of liquids).

The formulations can also be administered using other means, for example, rectal administration. Formulations useful for rectal administration, such as suppositories, are well known to those of skill in the art. The compounds can also be administered by inhalation (e.g., in the form of an aerosol either nasally or using delivery articles of the type set forth in U.S. Pat. No. 4,922,901 to Brooks et al., the disclosure of which is incorporated herein in its entirety); topically (e.g., in lotion form); transdermally (e.g., using a transdermal patch) or iontophoretically; or by sublingual or buccal administration. Although it is possible to administer the compounds in the form of a bulk active chemical, it is preferred to present each compound in the form of a pharmaceutical composition or formulation for efficient and effective administration.

Exemplary methods for administering such compounds will be apparent to the skilled artisan. The usefulness of these formulations can depend on the particular composition used and the particular subject receiving the treatment. These formulations can contain a liquid carrier that can be oily, aqueous, emulsified or contain certain solvents suitable to the mode of administration.

The compositions can be administered intermittently or at a gradual, continuous, constant or controlled rate to a warm-blooded animal (e.g., a mammal such as a mouse, rat, cat, rabbit, dog, pig, cow, or monkey), but advantageously are administered to a human being. In addition, the time of day and the number of times per day that the pharmaceutical formulation is administered can vary. Other suitable methods for administering the compounds of the present invention are described in U.S. Pat. No. 5,604,231 to Smith et al., the contents of which are hereby incorporated by reference.

In an embodiment of the present invention and as will be appreciated by those skilled in the art, the compound of the present invention may be administered in combination with other therapeutic compounds. For example, a compound of this invention can be used in combination with other NNR ligands (such as varenicline), antioxidants (such as free radical scavenging agents), antibacterial agents (such as penicillin antibiotics), antiviral agents (such as nucleoside analogs, like zidovudine and acyclovir), anticoagulants (such as warfarin), anti-inflammatory agents (such as NSAIDs), anti-pyretics, analgesics, anesthetics (such as used in surgery), acetylcholinesterase inhibitors (such as donepezil and galantamine), antipsychotics (such as haloperidol, clozapine, olanzapine, and quetiapine), immuno-suppressants (such as cyclosporin and methotrexate), neuroprotective agents, steroids (such as steroid hormones), corticosteroids (such as dexamethasone, predisone, and hydrocortisone), vitamins, minerals, nutraceuticals, anti-depressants (such as imipramine, fluoxetine, paroxetine, escitalopram, sertraline, venlafaxine, and duloxetine), anxiolytics (such as alprazolam and buspirone), anticonvulsants (such as phenytoin and gabapentin), vasodilators (such as prazosin and sildenafil), mood stabilizers (such as valproate and aripiprazole), anti-cancer drugs (such as anti-proliferatives), antihypertensive agents (such as atenolol, clonidine, amlopidine, verapamil, and olmesartan), laxatives, stool softeners, diuretics (such as furosemide), anti-spasmotics (such as dicyclomine), anti-dyskinetic agents, and anti-ulcer medications (such as esomeprazole).

The compounds of the present invention may be employed alone or in combination with other therapeutic agents, including other compounds of the present invention. Such a combination of pharmaceutically active agents may be administered together or separately and, when administered separately, administration may occur simultaneously or sequentially, in any order. The amounts of the compounds or agents and the relative timings of administration will be selected in order to achieve the desired therapeutic effect. The administration in combination of a compound of the formulae of the present invention including salts or solvates thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time. The compounds of the present invention may be used in the treatment of a variety of disorders and conditions and, as such, the compounds of the present invention may be used in combination with a variety of other suitable therapeutic agents useful in the treatment or prophylaxis of those disorders or conditions.

The following examples are provided to illustrate the present invention, and should not be construed as limiting thereof. In these examples, all parts and percentages are by weight, unless otherwise noted.

The appropriate dose of the compound is that amount effective to prevent occurrence of the symptoms of the disorder or to treat some symptoms of the disorder from which the patient suffers. By “effective amount”, “therapeutic amount” or “effective dose” is meant that amount sufficient to elicit the desired pharmacological or therapeutic effects, thus resulting in effective prevention or treatment of the disorder.

When treating a CNS disorder, an effective amount of compound is an amount sufficient to pass across the blood-brain barrier of the subject, to bind to relevant receptor sites in the brain of the subject and to modulate the activity of relevant NNR subtypes (e.g., provide neurotransmitter secretion, thus resulting in effective prevention or treatment of the disorder). Prevention of the disorder is manifested by delaying the onset of the symptoms of the disorder. Treatment of the disorder is manifested by a decrease in the symptoms associated with the disorder or an amelioration of the recurrence of the symptoms of the disorder. Preferably, the effective amount is sufficient to obtain the desired result, but insufficient to cause appreciable side effects.

The effective dose can vary, depending upon factors such as the condition of the patient, the severity of the symptoms of the disorder, and the manner in which the pharmaceutical composition is administered. For human patients, the effective dose of typical compounds generally requires administering the compound in an amount sufficient to modulate the activity of relevant NNRs, but the amount should be insufficient to induce effects on skeletal muscles and ganglia to any significant degree. The effective dose of compounds will of course differ from patient to patient, but in general includes amounts starting where CNS effects or other desired therapeutic effects occur but below the amount where muscular effects are observed.

The compounds described herein, when employed in effective amounts in accordance with the methods described herein, can provide some degree of prevention of the progression of, ameliorate symptoms of, and ameliorate to some degree of the recurrence of CNS or other disorders. The effective amounts of those compounds are typically below the threshold concentration required to elicit any appreciable side effects, for example those effects relating to skeletal muscle or ganglia. The compounds can be administered in a therapeutic window in which certain CNS and other disorders are treated and certain side effects are avoided. Ideally, the effective dose of the compounds described herein is sufficient to provide the desired effects upon the disorder but is insufficient (i.e., is not at a high enough level) to provide undesirable side effects. Preferably, the compounds are administered at a dosage effective for treating the CNS or other disorders but less than ⅕, and often less than 1/10, the amount required to elicit certain side effects to any significant degree.

Most preferably, effective doses are at very low concentrations, where maximal effects are observed to occur, with a minimum of side effects. An effective dose of such compounds may require administering the compound in an amount of less than 5 mg/kg of patient weight. The compounds of the present invention may be administered in an amount from less than about 1 mg/kg patent weight and usually less than about 100 μg/kg of patient weight, but may be between about 10 μg to less than 100 μg/kg of patient weight. The foregoing doses typically represent that amount administered as a single dose, or as one or more doses administered over a 24-hour period.

For human patients, an effective dose of typical compounds generally requires administering the compound in an amount of at least about 1, often at least about 10, and frequently at least about 100 mg/24 hr/patient. For human patients, an effective dose of typical compounds requires administering the compound which generally does not exceed about 500, often does not exceed about 400, and frequently does not exceed about 300 mg/24 hr/patient. In addition, the compositions may be advantageously administered at an effective dose such that the concentration of the compound within the plasma of the patient normally does not exceed 50 ng/mL, often does not exceed 30 ng/mL, and frequently does not exceed 10 ng/mL.

### IV. METHOD OF USING PHARMACEUTICAL COMPOSITIONS

The compounds of the present invention can be used for the prevention or treatment of various conditions or disorders for which other types of nicotinic compounds have been proposed or are shown to be useful as therapeutics, such as CNS disorders, inflammation, inflammatory response associated with bacterial and/or viral infection, pain, metabolic syndrome, autoimmune disorders, addictions, obesity or other disorders described in further detail herein. This compound can also be used as a diagnostic agent in receptor binding studies (in vitro and in vivo). Such therapeutic and other teachings are described, for example, in references previously listed herein, including Williams et al., Drug News Perspec. 7(4): 205 (1994), Arneric et al., CNS Drug Rev. 1(1): 1-26 (1995), Arneric et al., Exp. Opin. Invest. Drugs 5(1): 79-100 (1996), Bencherif et al., J. Pharmacol. Exp. Ther. 279: 1413 (1996), Lippiello et al., J. Pharmacol. Exp. Ther. 279: 1422 (1996), Damaj et al., J. Pharmacol. Exp. Ther. 291: 390 (1999); Chiari et al., Anesthesiology 91: 1447 (1999), Lavand'homme and Eisenbach, Anesthesiology 91: 1455 (1999), Holladay et al., J. Med. Chem. 40(28): 4169-94 (1997), Bannon et al., Science 279: 77 (1998), PCT WO 94/08992, PCT WO 96/31475, PCT WO 96/40682, and U.S. Pat. No. 5,583,140 to Bencherif et al., U.S. Pat. No. 5,597,919 to Dull et al., U.S. Pat. No. 5,604,231 to Smith et al. and U.S. Pat. No. 5,852,041 to Cosford et al.

### CNS Disorders

The compounds and their pharmaceutical compositions are useful in the treatment or prevention of a variety of CNS disorders, including neurodegenerative disorders, neuropsychiatric disorders, neurologic disorders, and addictions. The compounds and their pharmaceutical compositions can be used to treat or prevent cognitive deficits and dysfunctions, age-related and otherwise; attentional disorders and dementias, including those due to infectious agents or metabolic disturbances; to provide neuroprotection; to treat convulsions and multiple cerebral infarcts; to treat mood disorders, compulsions and addictive behaviors; to provide analgesia; to control inflammation, such as mediated by cytokines and nuclear factor kappa B; to treat inflammatory disorders; to provide pain relief; and to treat infections, as anti-infectious agents for treating bacterial, fungal, and viral infections. Among the disorders, diseases and conditions that the compounds and pharmaceutical compositions of the present invention can be used to treat or prevent are: age-associated memory impairment (AAMI), mild cognitive impairment (MCI), age-related cognitive decline (ARCD), pre-senile dementia, early onset Alzheimer's disease, senile dementia, dementia of the Alzheimer's type, Alzheimer's disease, cognitive impairment no dementia (CIND), Lewy body dementia, HIV-dementia, AIDS dementia complex, vascular dementia, Down syndrome, head trauma, traumatic brain injury (TBI), dementia pugilistica, Creutzfeld-Jacob Disease and prion diseases, stroke, central ischemia, peripheral ischemia, attention deficit disorder, attention deficit hyperactivity disorder, dyslexia, schizophrenia, schizophreniform disorder, schizoaffective disorder, cognitive dysfunction in schizophrenia, cognitive deficits in schizophrenia, Parkinsonism including Parkinson's disease, postencephalitic parkinsonism, parkinsonism-dementia of Gaum, frontotemporal dementia Parkinson's Type (FTDP), Pick's disease, Niemann-Pick's Disease, Huntington's Disease, Huntington's chorea, tardive dyskinesia, hyperkinesia, progressive supranuclear palsy, progressive supranuclear paresis, restless leg syndrome, Creutzfeld-Jakob disease, multiple sclerosis, amyotrophic lateral sclerosis (ALS), motor neuron diseases (MND), multiple system atrophy (MSA), corticobasal degeneration, Guillain-Barré Syndrome (GBS), and chronic inflammatory demyelinating polyneuropathy (CIDP), epilepsy, autosomal dominant nocturnal frontal lobe epilepsy, mania, anxiety, depression, premenstrual dysphoria, panic disorders, bulimia, anorexia, narcolepsy, excessive daytime sleepiness, bipolar disorders, generalized anxiety disorder, obsessive compulsive disorder, rage outbursts, conduct disorder, oppositional defiant disorder, Tourette's syndrome, autism, drug and alcohol addiction, tobacco addiction, obesity, cachexia, psoriasis, lupus, acute cholangitis, aphthous stomatitis, ulcers, asthma, ulcerative colitis, inflammatory bowel disease, Crohn's disease, irritable bowel syndrome, spastic dystonia, diarrhea, constipation, pouchitis, viral pneumonitis, arthritis, including, rheumatoid arthritis and osteoarthritis, endotoxaemia, sepsis, atherosclerosis, idiopathic pulmonary fibrosis, acute pain, chronic pain, neuropathies, urinary incontinence, diabetes, sexual dysfunction, neoplasias, and preeclampsia.

Cognitive impairments or dysfunctions may be associated with psychiatric disorders or conditions, such as schizophrenia and other psychotic disorders, including but not limited to psychotic disorder, schizophreniform disorder, schizoaffective disorder, delusional disorder, brief psychotic disorder, shared psychotic disorder, and psychotic disorders due to a general medical conditions, dementias and other cognitive disorders, including but not limited to mild cognitive impairment, pre-senile dementia, Alzheimer's disease, senile dementia, dementia of the Alzheimer's type, age-related memory impairment, Lewy body dementia, vascular dementia, AIDS dementia complex, dyslexia, Parkinsonism including Parkinson's disease, cognitive impairment and dementia of Parkinson's Disease, cognitive impairment of multiple sclerosis, cognitive impairment caused by traumatic brain injury, dementias due to other general medical conditions, anxiety disorders, including but not limited to panic disorder without agoraphobia, panic disorder with agoraphobia, agoraphobia without history of panic disorder, specific phobia, social phobia, obsessive-compulsive disorder, post-traumatic stress disorder, acute stress disorder, generalized anxiety disorder and generalized anxiety disorder due to a general medical condition, mood disorders, including but not limited to major depressive disorder, dysthymic disorder, bipolar depression, bipolar mania, bipolar I disorder, depression associated with manic, depressive or mixed episodes, bipolar II disorder, cyclothymic disorder, and mood disorders due to general medical conditions, sleep disorders, including but not limited to dyssomnia disorders, primary insomnia, primary hypersomnia, narcolepsy, parasomnia disorders, nightmare disorder, sleep terror disorder and sleepwalking disorder, mental retardation, learning disorders, motor skills disorders, communication disorders, pervasive developmental disorders, attention-deficit and disruptive behavior disorders, attention deficit disorder, attention deficit hyperactivity disorder, feeding and eating disorders of infancy, childhood, or adults, tic disorders, elimination disorders, substance-related disorders, including but not limited to substance dependence, substance abuse, substance intoxication, substance withdrawal, alcohol-related disorders, amphetamine or amphetamine-like-related disorders, caffeine-related disorders, cannabis-related disorders, cocaine-related disorders, hallucinogen-related disorders, inhalant-related disorders, nicotine-related disorders, opioid-related disorders, phencyclidine or phencyclidine-like-related disorders, and sedative-, hypnotic- or anxiolytic-related disorders, personality disorders, including but not limited to obsessive-compulsive personality disorder and impulse-control disorders.

Cognitive performance may be assessed with a validated cognitive scale, such as, for example, the cognitive subscale of the Alzheimer's Disease Assessment Scale (ADAS-cog). One measure of the effectiveness of the compounds of the present invention in improving cognition may include measuring a patient's degree of change according to such a scale.

Regarding compulsions and addictive behaviors, the compounds of the present invention may be used as a therapy for nicotine addiction and for other brain-reward disorders, such as substance abuse including alcohol addiction, illicit and prescription drug addiction, eating disorders, including obesity, and behavioral addictions, such as gambling, or other similar behavioral manifestations of addiction.

The above conditions and disorders are discussed in further detail, for example, in the American Psychiatric Association: Diagnostic and Statistical Manual of Mental Disorders, Fourth Edition, Text Revision, Washington, D.C., American Psychiatric Association, 2000. This Manual may also be referred to for greater detail on the symptoms and diagnostic features associated with substance use, abuse, and dependence.

Preferably, the treatment or prevention of diseases, disorders and conditions occurs without appreciable adverse side effects, including, for example, significant increases in blood pressure and heart rate, significant negative effects upon the gastro-intestinal tract, and significant effects upon skeletal muscle.

The compounds of the present invention, when employed in effective amounts, are believed to modulate the activity of the α4β2 and α7 NNRs without appreciable interaction with the nicotinic subtypes that characterize the human ganglia, as demonstrated by a lack of the ability to elicit nicotinic function in adrenal chromaffin tissue, or skeletal muscle, further demonstrated by a lack of the ability to elicit nicotinic function in cell preparations expressing muscle-type nicotinic receptors. Thus, these compounds are believed capable of treating or preventing diseases, disorders and conditions without eliciting significant side effects associated activity at ganglionic and neuromuscular sites. Thus, administration of the compounds is believed to provide a therapeutic window in which treatment of certain diseases, disorders and conditions is provided, and certain side effects are avoided. That is, an effective dose of the compound is believed sufficient to provide the desired effects upon the disease, disorder or condition, but is believed insufficient, namely is not at a high enough level, to provide undesirable side effects.

Thus, the present invention provides the use of a compound of the present invention, or a pharmaceutically acceptable salt thereof, for use in therapy, such as a therapy described above.

In yet another aspect the present invention provides the use of a compound of the present invention, or a pharmaceutically acceptable salt thereof, in the manufacture of a medicament for use in the treatment of a CNS disorder, such as a disorder, disease or condition described hereinabove.

### Inflammation

The nervous system, primarily through the vagus nerve, is known to regulate the magnitude of the innate immune response by inhibiting the release of macrophage tumor necrosis factor (TNF). This physiological mechanism is known as the “cholinergic anti-inflammatory pathway” (see, for example, Tracey, “The Inflammatory Reflex,” Nature 420:853-9 (2002)). Excessive inflammation and tumor necrosis factor synthesis cause morbidity and even mortality in a variety of diseases. These diseases include, but are not limited to, endotoxemia, rheumatoid arthritis, osteoarthritis, psoriasis, asthma, atherosclerosis, idiopathic pulmonary fibrosis, and inflammatory bowel disease.

Inflammatory conditions that can be treated or prevented by administering the compounds described herein include, but are not limited to, chronic and acute inflammation, psoriasis, endotoxemia, gout, acute pseudogout, acute gouty arthritis, arthritis, rheumatoid arthritis, osteoarthritis, allograft rejection, chronic transplant rejection, asthma, atherosclerosis, mononuclear-phagocyte dependent lung injury, idiopathic pulmonary fibrosis, atopic dermatitis, chronic obstructive pulmonary disease, adult respiratory distress syndrome, acute chest syndrome in sickle cell disease, inflammatory bowel disease, Crohn's disease, ulcerative colitis, acute cholangitis, aphteous stomatitis, pouchitis, glomerulonephritis, lupus nephritis, thrombosis, and graft vs. host reaction.

Inflammatory Response Associated with Bacterial and/or Viral Infection

Many bacterial and/or viral infections are associated with side effects brought on by the formation of toxins, and the body's natural response to the bacteria or virus and/or the toxins. As discussed above, the body's response to infection often involves generating a significant amount of TNF and/or other cytokines. The over-expression of these cytokines can result in significant injury, such as septic shock (when the bacteria is sepsis), endotoxic shock, urosepsis and toxic shock syndrome.

Cytokine expression is mediated by NNRs, and can be inhibited by administering agonists or partial agonists of these receptors. Those compounds described herein that are agonists or partial agonists of these receptors can therefore be used to minimize the inflammatory response associated with bacterial infection, as well as viral and fungal infections. Examples of such bacterial infections include anthrax, botulism, and sepsis. Some of these compounds may also have antimicrobial properties.

These compounds can also be used as adjunct therapy in combination with existing therapies to manage bacterial, viral and fungal infections, such as antibiotics, antivirals and antifungals. Antitoxins can also be used to bind to toxins produced by the infectious agents and allow the bound toxins to pass through the body without generating an inflammatory response. Examples of antitoxins are disclosed, for example, in U.S. Pat. No. 6,310,043 to Bundle et al. Other agents effective against bacterial and other toxins can be effective and their therapeutic effect can be complemented by co-administration with the compounds described herein.

### Pain

The compounds can be administered to treat and/or prevent pain, including acute, neurologic, inflammatory, neuropathic and chronic pain. The compounds can be used in conjunction with opiates to minimize the likelihood of opiate addiction (e.g., morphine sparing therapy). The analgesic activity of compounds described herein can be demonstrated in models of persistent inflammatory pain and of neuropathic pain, performed as described in U.S. Published Patent Application No. 20010056084 A1 (Allgeier et al.) (e.g., mechanical hyperalgesia in the complete Freund's adjuvant rat model of inflammatory pain and mechanical hyperalgesia in the mouse partial sciatic nerve ligation model of neuropathic pain).

The analgesic effect is suitable for treating pain of various genesis or etiology, in particular in treating inflammatory pain and associated hyperalgesia, neuropathic pain and associated hyperalgesia, chronic pain (e.g., severe chronic pain, post-operative pain and pain associated with various conditions including cancer, angina, renal or biliary colic, menstruation, migraine, and gout). Inflammatory pain may be of diverse genesis, including arthritis and rheumatoid disease, teno-synovitis and vasculitis. Neuropathic pain includes trigeminal or herpetic neuralgia, diabetic neuropathy pain, causalgia, low back pain and deafferentation syndromes such as brachial plexus avulsion.

### Neovascularization

The α7 NNR is associated with neovascularization. Inhibition of neovascularization, for example, by administering antagonists (or at certain dosages, partial agonists) of the α7 NNR can treat or prevent conditions characterized by undesirable neovascularization or angiogenesis. Such conditions can include those characterized by inflammatory angiogenesis and/or ischemia-induced angiogenesis. Neovascularization associated with tumor growth can also be inhibited by administering those compounds described herein that function as antagonists or partial agonists of α7 NNR.

Specific antagonism of α7 NNR-specific activity reduces the angiogenic response to inflammation, ischemia, and neoplasia. Guidance regarding appropriate animal model systems for evaluating the compounds described herein can be found, for example, in Heeschen, C. et al., “A novel angiogenic pathway mediated by non-neuronal nicotinic acetylcholine receptors,” J. Clin. Invest. 110(4):527-36 (2002).

Representative tumor types that can be treated using the compounds described herein include NSCLC, ovarian cancer, pancreatic cancer, breast carcinoma, colon carcinoma, rectum carcinoma, lung carcinoma, oropharynx carcinoma, hypopharynx carcinoma, esophagus carcinoma, stomach carcinoma, pancreas carcinoma, liver carcinoma, gallbladder carcinoma, bile duct carcinoma, small intestine carcinoma, urinary tract carcinoma, kidney carcinoma, bladder carcinoma, urothelium carcinoma, female genital tract carcinoma, cervix carcinoma, uterus carcinoma, ovarian carcinoma, choriocarcinoma, gestational trophoblastic disease, male genital tract carcinoma, prostate carcinoma, seminal vesicles carcinoma, testes carcinoma, germ cell tumors, endocrine gland carcinoma, thyroid carcinoma, adrenal carcinoma, pituitary gland carcinoma, skin carcinoma, hemangiomas, melanomas, sarcomas, bone and soft tissue sarcoma, Kaposi's sarcoma, tumors of the brain, tumors of the nerves, tumors of the eyes, tumors of the meninges, astrocytomas, gliomas, glioblastomas, retinoblastomas, neuromas, neuroblastomas, Schwannomas, meningiomas, solid tumors arising from hematopoietic malignancies (such as leukemias, chloromas, plasmacytomas and the plaques and tumors of mycosis fungoides and cutaneous T-cell lymphoma/leukemia), and solid tumors arising from lymphomas.

The compounds can also be administered in conjunction with other forms of anti-cancer treatment, including co-administration with antineoplastic antitumor agents such as cis-platin, adriamycin, daunomycin, and the like, and/or anti-VEGF (vascular endothelial growth factor) agents, as such are known in the art.

The compounds can be administered in such a manner that they are targeted to the tumor site. For example, the compounds can be administered in microspheres, microparticles or liposomes conjugated to various antibodies that direct the microparticles to the tumor. Additionally, the compounds can be present in microspheres, microparticles or liposomes that are appropriately sized to pass through the arteries and veins, but lodge in capillary beds surrounding tumors and administer the compounds locally to the tumor. Such drug delivery devices are known in the art.

### Other Disorders

In addition to treating CNS disorders, inflammation, and neovascularization, and pain, the compounds of the present invention can be also used to prevent or treat certain other conditions, diseases, and disorders in which NNRs play a role. Examples include autoimmune disorders such as Lupus, disorders associated with cytokine release, cachexia secondary to infection (e.g., as occurs in AIDS, AIDS related complex and neoplasia), obesity, pemphitis, urinary incontinence, retinal diseases, infenctious diseases, myasthenia, Eaton-Lambert syndrome, hypertension, preeclampsia, osteoporosis, vasoconstriction, vasodilatation, cardiac arrhythmias, type I diabetes, bulimia, anorexia as well as those indications set forth in published PCT application WO 98/25619. The compounds of this invention can also be administered to treat convulsions such as those that are symptomatic of epilepsy, and to treat conditions such as syphillis and Creutzfeld-Jakob disease.

### Diagnostic Uses

The compounds can be used in diagnostic compositions, such as probes, particularly when they are modified to include appropriate labels. The probes can be used, for example, to determine the relative number and/or function of specific receptors, particularly the α4β2 and α7 receptor subtypes. For this purpose the compounds of the present invention most preferably are labeled with a radioactive isotopic moiety such as ¹¹C, ¹⁸F, ⁷⁶Br, ¹²³I or ¹²⁵I.

The administered compounds can be detected using known detection methods appropriate for the label used. Examples of detection methods include position emission topography (PET) and single-photon emission computed tomography (SPECT). The radiolabels described above are useful in PET (e.g., ¹¹C, ¹⁸F or ⁷⁶Br) and SPECT (e.g., ¹²³I) imaging, with half-lives of about 20.4 minutes for ¹¹C, about 109 minutes for ¹⁸F, about 13 hours for ¹²³I, and about 16 hours for ⁷⁶Br. A high specific activity is desired to visualize the selected receptor subtypes at non-saturating concentrations. The administered doses typically are below the toxic range and provide high contrast images. The compounds are expected to be capable of administration in non-toxic levels. Determination of dose is carried out in a manner known to one skilled in the art of radiolabel imaging. See, for example, U.S. Pat. No. 5,969,144 to London et al.

The compounds can be administered using known techniques. See, for example, U.S. Pat. No. 5,969,144 to London et al., as noted. The compounds can be administered in formulation compositions that incorporate other ingredients, such as those types of ingredients that are useful in formulating a diagnostic composition. Compounds useful in accordance with carrying out the present invention most preferably are employed in forms of high purity. See, U.S. Pat. No. 5,853,696 to Elmalch et al.

After the compounds are administered to a subject (e.g., a human subject), the presence of that compound within the subject can be imaged and quantified by appropriate techniques in order to indicate the presence, quantity, and functionality of selected NNR subtypes. In addition to humans, the compounds can also be administered to animals, such as mice, rats, dogs, and monkeys. SPECT and PET imaging can be carried out using any appropriate technique and apparatus. See Villemagne et al., In: Arneric et al. (Eds.) Neuronal Nicotinic Receptors: Pharmacology and Therapeutic Opportunities, 235-250 (1998) and U.S. Pat. No. 5,853,696 to Elmalch et al., each herein incporated by reference, for a disclosure of representative imaging techniques.

The radiolabeled compounds bind with high affinity to selective NNR subtypes (e.g., α4β2, α7) and preferably exhibit negligible non-specific binding to other nicotinic cholinergic receptor subtypes (e.g., those receptor subtypes associated with muscle and ganglia). As such, the compounds can be used as agents for noninvasive imaging of nicotinic cholinergic receptor subtypes within the body of a subject, particularly within the brain for diagnosis associated with a variety of CNS diseases and disorders.

In one aspect, the diagnostic compositions can be used in a method to diagnose disease in a subject, such as a human patient. The method involves administering to that patient a detectably labeled compound as described herein, and detecting the binding of that compound to selected NNR subtypes (e.g., α4β2 and α7 receptor subtypes). Those skilled in the art of using diagnostic tools, such as PET and SPECT, can use the radiolabeled compounds described herein to diagnose a wide variety of conditions and disorders, including conditions and disorders associated with dysfunction of the central and autonomic nervous systems. Such disorders include a wide variety of CNS diseases and disorders, including Alzheimer's disease, Parkinson's disease, and schizophrenia. These and other representative diseases and disorders that can be evaluated include those that are set forth in U.S. Pat. No. 5,952,339 to Bencherif et al.

In another aspect, the diagnostic compositions can be used in a method to monitor selective nicotinic receptor subtypes of a subject, such as a human patient. The method involves administering a detectably labeled compound as described herein to that patient and detecting the binding of that compound to selected nicotinic receptor subtypes namely, the α4β2 and α7 receptor subtypes.

### Receptor Binding

The compounds of this invention can be used as reference ligands in binding assays for compounds which bind to NNR subtypes, particularly the α4β2 and α7 receptor subtypes. For this purpose the compounds of this invention are preferably labeled with a radioactive isotopic moiety such as ³H, or ¹⁴C. Examples of such binding assays are described in detail below.

### V. SYNTHETIC EXAMPLES

### Example 1

Example 1 is the synthesis of 9-oxa-3,7-diazabicyclo[3.3.1]nonane, suitably protected (preferably with either a Boc or a Cbz group) for use in N-aryl coupling reactions.

### N-(Benzyloxycarbonyl)diallylamine

Benzyl chloroformate (0.33 mol, 50 mL) was added to a solution of diallylamine (0.30 mol, 37 mL) and triethylamine (0.33 mol, 46 mL) in dichloromethane (300 mL). The reaction mixture was allowed to stir at ambient temperature overnight. The mixture was washed with water (4×75 mL), and the organic phase was separated, dried over magnesium sulfate, and concentrated by rotary evaporation to give a light brown oil. The oil was purified by silica gel flash chromatography (3:1 hexanes/ethyl acetate) to yield 47 g (68%) of N-benzyloxycarbonyl diallylamine as a colorless oil.

### N-(Benzyloxycarbonyl)-2,6-bis(mercurylmethyl)morpholine diacetate

To a solution of mercury(II) acetate (0.130 mol, 41.4 g) in water (120 mL) was added N-(benzyloxycarbonyl)diallylamine (0.065 mol, 15 g). The solution was allowed to stir for 24 h, during which a colorless precipitate appeared. The water was removed by rotary evaporation and the residue washed with ethanol and dried under vacuum to yield 40.6 g (81.6%) of N-(benzyloxycarbonyl)-2,6-bis(mercurylmethyl)morpholine diacetate.

### N-(Benzyloxycarbonyl)-2,6-bis(iodomethyl)morpholine

N-Benzyloxycarbonyl-2,6-bis(mercurylmethyl)morpholine diacetate (0.0530 mol, 40.6 g) was added to a solution of iodine (0.1589 mol, 40.36 g) in chloroform (250 mL). Water (120 mL) was added and the reaction mixture was stirred under reflux for 14 h. The solution was filtered to remove the resulting red precipitate. The filtrate was washed sequentially with aqueous sodium thiosulfate and water, dried over anhydrous calcium chloride and concentrated to yield mixture of thick brown oil and solid (˜30 g). This residue was recrystallized from ethanol to yield 22.3 g (84%) of N-(benzyloxycarbonyl)-2,6-bis(iodomethyl)morpholine.

### 3-(Benzyloxycarbonyl)-9-oxa-3,7-diazabicyclo[3.3.1]nonane

N-(Benzyloxycarbonyl)-2,6-bis(iodomethyl)morpholine (0.01 mol, 5.0 g) was dissolved in 7 N methanolic ammonia (40 mL), and heated at 150° C. for 15 min in a microwave at 200 psi. The mixture was concentrated and the residue washed with water and purified by HPLC to yield 1.0 g (39%) of 3-(benzyloxycarbonyl)-9-oxa-3,7-diazabicyclo[3.3.1]nonane.

### 3-(Benzyloxycarbonyl)-7-(t-butoxycarbonyl)-9-oxa-3,7-diazabicyclo[3.3.1]nonane

3-(Benzyloxycarbonyl)-9-oxa-3,7-diazabicyclo[3.3.1]nonane (0.0156 mol, 4.08 g) was dissolved in dry dichloromethane (45 mL). Triethylamine (3.5 mL) and di-t-butyl dicarbonate (0.0188 mol, 4.09 g) were added to the solution. The reaction mixture was allowed to stir at room temperature overnight. The mixture was washed with water (4×10 mL), dried over magnesium sulfate and concentrated to yield 5.4 g, (96%) of 3-(benzyloxycarbonyl)-7-(t-butoxycarbonyl)-9-oxa-3,7-diazabicyclo[3.3.1]nonane.

### 3-(t-Butoxycarbonyl)-9-oxa-3,7-diazabicyclo[3.3.1]nonane

3-(Benzyloxycarbonyl)-7-(t-butoxycarbonyl)-9-oxa-3,7-diazabicyclo[3.3.1]nonane (0.0041 mol, 1.5 g) in methanol (80 mL) was hydrogenated over palladium hydroxide. The solution filtered and concentrated to give 1.2 g of crude product. This was dissolved in ethanol and purified by HPLC to yield 0.36 g (38%) of 3-t-butoxycarbonyl-9-oxa-3,7-diazabicyclo[3.3.1]nonane.

### Examples 2-4

Examples 2-4 involve coupling reactions of 3-(t-butoxycarbonyl)-9-oxa-3,7-diazabicyclo[3.3.1]nonane with various aryl halides. As will be appreciated by those skilled in the art, in some cases, such coupling reactions are palladium catalyzed; in other cases (such as example 3), no palladium catalyst is necessary, as some aryl halides are sufficiently reactive toward nucleophilic substitution such that the coupling can be accomplished without catalysis.

### Example 2

### 3-(5-Fluoropyridin-3-yl)-9-oxa-3,7-diazabicyclo[3.3.1]nonane trifluoroacetate

5-Bromo-3-fluoropyridine (1.14 g, 6.48 mmol) and 3-(t-butyloxycarbonyl)-9-oxa-3,7-diazabicyclo[3.3.1]nonane (1.14 g, 5.00 mmol) were combined in dry toluene (45 mL), followed by addition of tris(dibenzylideneacetone)dipalladium (91.6 mg, 0.100 mmol), 4,5-bis(diphenylphophino)-9,9-dimethylxanthene (174 mg, 0.301 mmol), and sodium t-butoxide (721 mg, 7.51 mmol). The reaction vessel was flushed with argon and the reaction solution was allowed to stir at 95° C. for 3 hours. The reaction mixture was cooled to ambient temperature, diluted with ethyl acetate (30 mL), and washed with water (10 mL). The organic layer was separated and concentrated under reduced pressure. The residue was purified by HPLC to yield 3-(t-butoxycarbonyl)-7-(5-fluoropyridin-3-yl)-9-oxa-3,7-diazabicyclo[3.3.1]nonane (0.81 g, yield 50%). This was dissolved in dichloromethane/trifluoroacetic acid (1:1) (3 mL) and stirred for 1 h. The reaction solution was concentrated under vacuum to yield 3-(5-fluoropyridin-3-yl)-9-oxa-3,7-diazabicyclo[3.3.1]nonane trifluoroacetate (0.84 g, 99%). ¹H NMR (CD₃OD) (δ) ppm: 3.22 (d, 2H), 3.5 (s, 4H), 3.70 (d, 2H), 4.3 (m, 2H), 7.2 (d, 1H), 7.9 (s, 1H), and 8.05 (s, 1H). LCMS: 224 (M+1).

Similar methodology was used to prepare 3-(5-cyanopyridin-3-yl)-9-oxa-3,7-diazabicyclo[3.3.1]nonane hemigalactarate: ¹H NMR (D₂O) (δ) ppm: 8.44 (d, 1H), 8.31 (s, 1H), 7.71 (s, 1H), 4.28 (s, 2H), 3.73 (d, 2H), 3.75 (m, 4H), 3.25 (m, 2H).

### Example 3

### 3-(6-Chloropyridazin-3-yl)-9-oxa-3,7-diazabicyclo[3.3.1]nonane trifluoroacetate

3,6-Dichloropyridazine (44.7 mg, 0.300 mmol) and 3-(t-butoxycarbonyl)-9-oxa-3,7-diazabicyclo[3.3.1]nonane (68.5 mg, 0.300 mmol) were combined in dry toluene (3 mL). Triethylamine (0.1 mL) was added and the reaction mixture was stirred at 80° C. for 4 hours. The reaction mixture was cooled to ambient temperature, diluted with ethyl acetate (5 mL), and washed with water (3×3 mL). Organic layer was separated and concentrated under reduced pressure. The residue was purified by HPLC to yield 3-(t-butyloxycarbonyl)-7-(6-chloropyridazin-3-yl)-9-oxa-3,7-diazabicyclo[3.3.1]nonane 0.061 g (60%). This was dissolved in dichloromethane:trifluoroacetic acid (1:1) (1 mL) and stirred at ambient temperature for 1 h. The reaction solution was concentrated under vacuum to yield 3-(6-chloropyridazin-3-yl)-9-oxa-3,7-diazabicyclo[3.3.1]nonane trifluoroacetate 0.063 g (99%). LCMS: 241(M+1).

### Example 4

### 3-(5-Methoxypyridin-3-yl)-9-oxa-3,7-diazabicyclo[3.3.1]nonane trifluoroacetate

3-Bromo-5-methoxypyridine (56.4 mg, 0.300 mmol) and 3-(t-butoxycarbonyl)-9-oxa-3,7-diazabicyclo[3.3.1]nonane (68.5 mg, 0.300 mmol) were combined in dry toluene (3 ml), followed by addition of tris(dibenzylideneacetone)dipalladium (13.7 mg, 0.0150 mmol), 2,2′-bis(diphenylphosphino)-1,1′-binaphthyl (18.7 mg, 0.0300 mmol), and sodium t-butoxide (115 mg, 1.20 mmol). The reaction vessel was flushed with argon and the reaction solution was stirred at 95° C. overnight. The reaction mixture was cooled to ambient temperature, diluted with ethyl acetate (5 mL), and washed with water (2×3 mL). The organic layer was separated and concentrated under reduced pressure. The residue was purified by HPLC to yield 3-(t-butyloxycarbonyl)-7-(5-methoxypyridin-3-yl)-9-oxa-3,7-diazabicyclo[3.3.1]nonane (0.055 g, 55%). This was dissolved in 1 mL of dichloromethane:trifluoroacetic acid (1:1) and stirred for 1 h. The reaction solution was concentrated under vacuum to yield 3-(5-methoxypyridin-3-yl)-9-oxa-3,7-diazabicyclo[3.3.1]nonane trifluoroacetate (0.057 g, 99%). LCMS: 236 (M+1).

### Example 5

### 3-(5,6-Dichloropyridin-3-yl)-9-oxa-3,7-diazabicyclo[3.3.1]nonane trifluoroacetate

3-Bromo-5,6-dichloropyridine (68 mg, 0.3 mmol) and 3-(t-butyloxycarbonyl)-9-oxa-3,7-diazabicyclo[3.3.1]nonane (68.49 mg, 0.3 mmol) were combined in dry toluene (3 ml), followed by addition of tris(dibenzylideneacetone)dipalladium (13.7 mg, 0.015 mmol), 2,2′-bis(diphenylphosphino)-1,1′-binaphthyl (18.7 mg, 0.03 mmol), and sodium t-butoxide (115.1 mg, 1.2 mmol). The reaction vessel was flushed with argon and the reaction solution was stirred at 95° C. overnight. The reaction mixture was cooled to ambient temperature, diluted with ethyl acetate (5 mL), and washed with water (2×3 mL). The organic layer was separated, and was concentrated under reduced pressure. The residue was purified by HPLC to yield 3-(t-butyloxycarbonyl)-7-(5,6-dichloropyridin-3-yl)-9-oxa-3,7-diazabicyclo[3.3.1]nonane (0.056g, 50%). The latter was dissolved 1 mL of 1:1 methylene chloride:trifluoroacetic acid and was stirred for 1 h. The reaction solution was concentrated under vacuum to yield 3-(5,6-dichloropyridin-3-yl)-9-oxa-3,7-diazabicyclo[3.3.1]nonane trifluoroacetate (0.057 g, 98%). LCMS: 274/276 (M/M+2).

### Example 6

### 5-Bromo-2-chloro-3-(4-fluorobutoxy)pyridine

To a solution of 5-bromo-2-chloro-3-hydroxypyridine (1.5 g, 7.20 mmol), fluorobutanol (7.92 mmol; 729.15 mg), triphenylphosphine (7.92 mmol; 2.10 g) in anhydrous tetrahydrofuran (20 ml), was added diisopropyl azodicarboxylate (7.92 mmoles; 1.67 mL; 1.70 g) dropwise at 0° C. The reaction mixture was stirred at room temperature overnight, concentrated, and the residue was purified by flash-chromatography to yield yellow oil. Yield 2 g (98%). ¹H NMR (δ) ppm: 8.05 (s, 1H), 7.36 (s, 1H), 4.61 (t, 1H), 4.50 (t, 1H), 4.12 (t, 2H), 2.01 (m, 4H).

### 3-[6-Chloro-5-(4-fluorobutoxy)pyridin-3-yl]-9-oxa-3,7-diazabicyclo[3.3.1]nonane trifluoroacetate

To a solution of 5-bromo-2-chloro-3-(4-fluorobutoxy)pyridine (56.4 mg, 0.300 mmol) and 3-(tert-butoxycarbonyl)-9-oxa-3,7-diazabicyclo[3.3.1]nonane (150 mg, 0.657 mmol) in dry toluene (7 ml) was added tris(dibenzylideneacetone)dipalladium (30 mg, 0.033 mmol), 4,5-bis(diphenylphosphino)-9,9-dimethylxanthene (57 mg, 0.099 mmol), and sodium tert-butoxide (95 mg, 0.986 mmol). The reaction vessel was flushed with argon and the reaction solution was stirred at 110° C. for 16 h. The reaction mixture was cooled to ambient temperature, filtered through a plug of diatomaceous earth. The filtrate was diluted with toluene and washed with water (2×3 mL). The organic layer was separated and concentrated under reduced pressure. The residue was purified by flash-chromatography to yield 3-(tert-butyloxycarbonyl)-7-[(4-fluorobutoxy)pyridin-3-yl]-9-oxa-3,7-diazabicyclo[3.3.1]nonane (0.054 g, 19%). This was dissolved in 1 mL of dichloromethane:trifluoroacetic acid (1:1) and stirred for 1 h. The reaction solution was concentrated under vacuum to yield 3-[6-chloro-5-(4-fluorobutoxy)pyridin-3-yl]-9-oxa-3,7-diazabicyclo[3.3.1]nonane trifluoroacetate (0.0179 g, 34%). ¹H NMR (CD₃OD) (δ) ppm: 7.64 (d, 1H), 7.20 (d, 1H), 4.59 (t, 1H), 4.43 (t, 1H), 4.24 (s, 2H), 4.12 (t, 2H), 3.90 (d, 2H), 3.57 (m, 4H), 3.22 (m, 2H), 1.96, (m, 4H). LCMS: 330, 332(M+1).

3-[6-Chloro-5-(4-fluoropropoxy)pyridin-3-yl]-9-oxa-3,7-diazabicyclo[3.3.1]nonane trifluoroacetate and 3-[6-chloro-5-(4-fluoroethoxy)pyridin-3-yl]-9-oxa-3,7-diazabicyclo[3.3.1]nonane trifluoroacetate were prepared via a modified version of the procedure described in Example 6.

3-(6-Chloro-5-(4-fluoropropoxy)pyridin-3-yl]-9-oxa-3,7-diazabicyclo[3.3.1]nonane trifluoroacetate ¹H NMR (CD₃OD) (δ) ppm: 7.78 (s, 1H), 7.22 (s, 1H), 4.75 (t, 1H), 4.60 (t, 1H), 4.25 (m, 4H), 3.80 (d, 2H), 3.60 (m, 4H), 3.30 (m, 2H), 2.28, (m, 2H).

3-[6-Chloro-5-(4-fluoroethoxy)pyridin-3-yl]-9-oxa-3,7-diazabicyclo[3.3.1]nonane trifluoroacetate ¹H NMR (CD₃OD) (δ) ppm: 7.76 (d, 1H), 7.26 (d, 1H), 4.83 (m, 1H), 4.78 (m, 1H), 4.40 (m, 1H), 4.33 (m, 1H), 4.27 (s, 2H), 3.80 (d, 2H), 3.56 (m, 4H), 3.27 (m, 2H).

### Example 7

### 3-(5-Cyclopropyl-6-chloropyridin-3-yl)-9-oxa-3,7-diazabicyclo[3.3.1]nonane trifluoroacetate

A solution of 7-(tert-butoxycarbonyl)-3-(5-bromo-6-chloropyridin-3-yl)-9-oxa-3,7-diazabicyclo[3.3.1]nonane (226 mg, 0.54 mmol), cyclopropyl boronic acid (60 mg, 0.70 mmol), tricyclohexylphosphine (15 mg, 0.40 mmol), potassium phosphate (401 mg, 1.89 mmol) and palladium acetate (6 mg, 0.027 mmol) in water (0.22 ml) and toluene (4.3 ml) was heated under argon with stirring at 100° C. for 16 h. The reaction mixture was filtered through diatomaceous earth, concentrated, and the residue purified by flash chromatography to give 122 mg (59.5%) of 7-(tert-butoxycarbonyl)-3-(5-cyclopropyl-6-chloropyridin-3-yl)-9-oxa-3,7-diazabicyclo[3.3.1]nonane. This material was treated with methylene chloride-trifluoroacetic acid (1 ml, 1:1) for 2 h at ambient temperature. The reaction mixture was concentrated, the residue was purified by preparative HPLC to give 3-(5-cyclopropyl-6-chloropyridin-3-yl)-9-oxa-3,7-diazabicyclo[3.3.1]nonane trifluoroacetate (44%): ¹H NMR (CD₃OD) (δ) ppm: 7.93 (s, 1H), 7.16 (s, 1H), 4.25 (s, 2H), 3.77 (d, 2H), 3.55 (m, 4H), 3.22 (d, 2H), 2.14 (m, 1H), 1.08 (m, 2H), 0.83 (m, 2H).

### VIII. BIOLOGICAL ASSAYS

### Example 8

### Characterization of Interactions at Nicotinic Acetylcholine Receptors

### Cell Lines

SH-EP1/human α4β2 (Eaton et al., 2003), SH-EP1/human α4β4 (Gentry et al., 2003), SH-EP1/α6β3β4α5 (Grinevich et al., 2005), TE671/RD and SH-SY5Y cell lines (obtained from Dr. Ron Lukas, Barrow Neurological Institute) were maintained in proliferative growth phase in Dulbecco's modified Eagle's medium (Gibco/BRL) with 10% horse serum (Gibco BRL), 5% fetal bovine serum (HyClone, Logan Utah), 1 mM sodium pyruvate, 4 mM L-glutamine. For maintenance of stable transfectants, the α4β2 and α4β4 cell media was supplemented with 0.25 mg/mL zeocin and 0.13 mg/mL hygromycin B. Selection was maintained for the α6β3β4α5 cells with 0.25 mg/mL of zeocin, 0.13 mg/mL of hygromycin B, 0.4 mg/mL of geneticin, and 0.2 mg/mL of blasticidin. HEK/human α7/RIC3 cells (obtained from J. Lindstrom, U. Pennsylvania) were maintained in proliferative growth phase in Dulbecco's modified Eagle's medium (Gibco/BRL) with 10% fetal bovine serum (HyClone, Logan Utah), 1 mM sodium pyruvate, 4 mM L-glutamine, 0.4 mg/mL geneticin; 0.2 mg/ml hygromycin B.

### Receptor Binding Assays

Preparation of membranes from rat tissues. Rat cortices were obtained from Analytical Biological Services, Incorporated (ABS, Wilmington, Del.). Tissues were dissected from female Sprague-Dawley rats, frozen and shipped on dry ice. Tissues were stored at −20° C. until needed for membrane preparation. Cortices from 10 rats were pooled and homogenized by Polytron (Kinematica GmbH, Switzerland) in 10 volumes (weight:volume) of ice-cold preparative buffer (KCl, 11 mM; KH₂PO₄, 6 mM; NaCl 137 mM; Na₂HPO₄ 8 mM; HEPES (free acid), 20 mM; iodoacetamide, 5 mM; EDTA, 1.5 mM; 0.1 mM PMSF pH 7.4). The resulting homogenate was centrifuged at 40,000 g for 20 minutes at 4° C. and the resulting pellet was resuspended in 20 volumes of ice-cold water. After 60-minute incubation at 4° C., a new pellet was collected by centrifugation at 40,000 g for 20 minutes at 4° C. The final pellet was resuspended in preparative buffer and stored at −20° C. On the day of the assay, tissue was thawed, centrifuged at 40,000 g for 20 minutes and then resuspended in PBS (Dulbecco's Phosphate Buffered Saline, Life Technologies, pH 7.4) to a final concentration of 2-3 mg protein/mL. Protein concentrations were determined using the Pierce BCA Protein Assay kit (Pierce Biotechnology, Rockford, Ill.), with bovine serum albumin as the standard. Preparation of membranes from clonal cell lines. Cells were harvested in ice-cold PBS, pH 7.4, then homogenized with a polytron (Brinkmann Instruments, Westbury, N.Y.). Homogenates were centrifuged at 40,000 g for 20 minutes (4° C.). The pellet was resuspended in PBS and protein concentration determined using the Pierce BCA Protein Assay kit (Pierce Biotechnology, Rockford, Ill.).

Competition binding to receptors in membrane preparations. Binding to nicotinic receptors was assayed on membranes using standard methods adapted from published procedures (Lippiello and Fernandes, 1986; Davies et al., 1999). In brief, membranes were reconstituted from frozen stocks (approximately 0.2 mg protein) and incubated for 2 h on ice in 150 ml assay buffer (PBS) in the presence of competitor compound (0.001 nM to 100 mM) and radioligand. [³H]-nicotine (L-(−)-[N-methyl-3H]-nicotine, 69.5 Ci/mmol, Perkin-Elmer Life Sciences) was used for human α4β2 binding studies. [³H]-epibatidine (52 Ci/mmol, Perkin-Elmer Life Sciences) was used for binding studies at the other receptor subtypes. Incubation was terminated by rapid filtration on a multimanifold tissue harvester (Brandel, Gaithersburg, Md.) using GF/B filters presoaked in 0.33% polyethyleneimine (w/v) to reduce non-specific binding. Filters were washed 3 times and the radioactivity retained was determined by liquid scintillation counting.

Binding data analysis. Binding data were expressed as percent total control binding. Replicates for each point were averaged and plotted against the log of drug concentration. The IC₅₀ (concentration of the compound that produces 50% inhibition of binding) was determined by least squares non-linear regression using GraphPad Prism software (GraphPAD, San Diego, Calif.). Kᵢ was calculated using the Cheng-Prusoff equation (Cheng and Prusoff, 1973).

### Example 9

### Tabular Spectral and Receptor Binding Data

The above illustrated amide coupling procedures were used as a basis to make the compounds shown in Table 1. Reagents and conditions will be readily apparent to those skilled in the art. In some cases, compounds were characterized by nuclear magnetic resonance (NMR) data. In other cases, compounds were structurally characterized by LCMS.

TABLE 1

| | LCMS | Rat | human | Rat α7 | Human |
|---|---|---|---|---|---|
| Structure | [M + H]⁺ | α4β2 Ki | α4β2 Ki | Ki | α7 Ki |
| 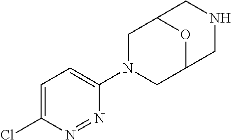 | 241 | 96 | 63 | | 1300 |
| 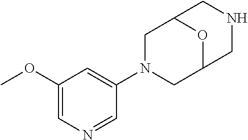 | 236 | 1.9 | 0.90 | 560 | 350 |
| 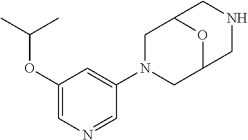 | 264 | 1.3 | 0.90 | 61000 | |
| 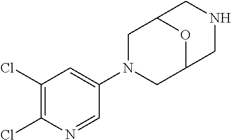 | 275 | 0.70 | 0.30 | 84 | 39 |
| 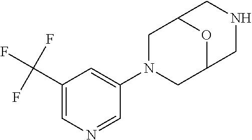 | 274 | 9.7 | 3.8 | 17000 | |
| 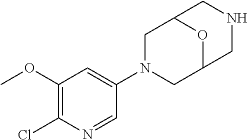 | 271 | 1.0 | 0.40 | 400 | 410 |
| 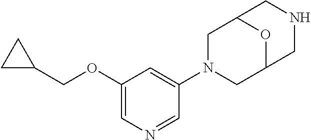 | 276 | 2.9 | 0.50 | 9300 | |
| 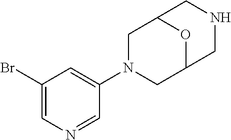 | 285 | 2.7 | 2.1 | 580 | 220 |
| 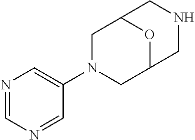 | 207 | 8.0 | 7.6 | 3200 | 2900 |
| 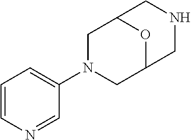 | 206 | 3.9 | 1.1 | 650 | 670 |
| 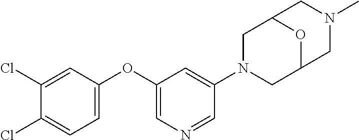 | 381 | 400 | 62 | | |
| 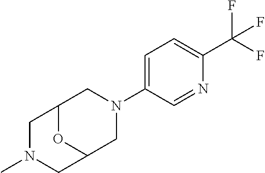 | 288 | | 1000 | | |
| 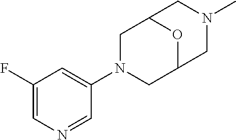 | 238 | 280 | 33 | | 2100 |
| 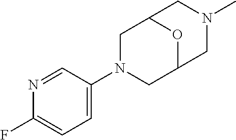 | 238 | 1100 | 140 | | |
| 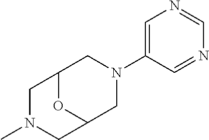 | 221 | | 270 | | |
| 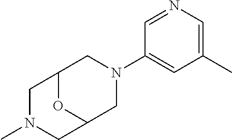 | 234 | 370 | 25 | | 8700 |
| 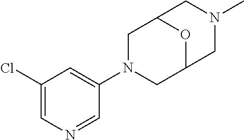 | 254 | 1600 | 77 | | |
| 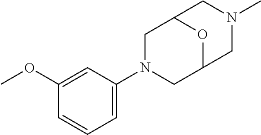 | 249 | | 1600 | | |
| 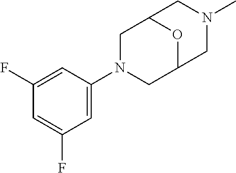 | 255 | 510 | 86 | | |
| 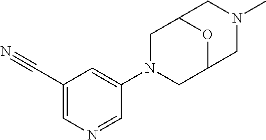 | 245 | | 260 | | |
| 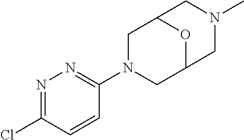 | 255 | | 29000 | | |
| 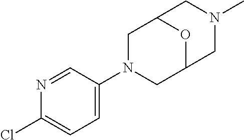 | 254 | 1800 | 120 | | |
| 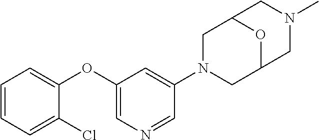 | 346 | 1200 | 84 | | |
| 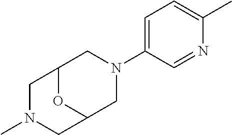 | 234 | 1700 | 150 | | |
| 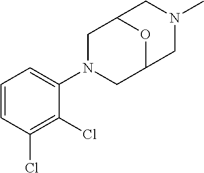 | 287 | | 5200 | | |
| 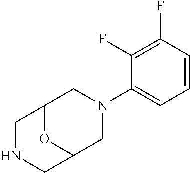 | 241 | | 260 | | |
| 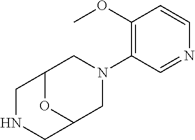 | 236 | 580 | 120 | | |
| 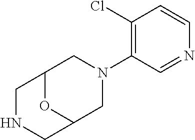 | 240 | | 250 | | |
| 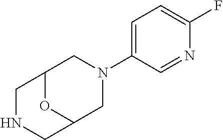 | 224 | 16 | 2.5 | 1400 | 180 |
| 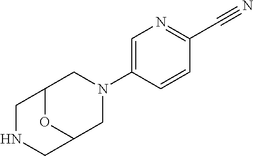 | 231 | 290 | 42 | | 8100 |
| 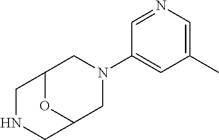 | 220 | 0.50 | 0.30 | 200 | 4500 |
| 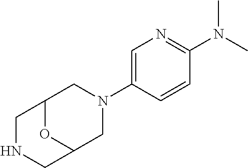 | 249 | | 1200 | | |
| 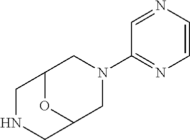 | 207 | 250 | 37 | | 2200 |
| 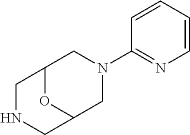 | 206 | 350 | 100 | | |
| 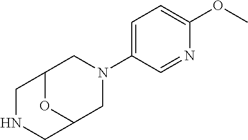 | 236 | | 200 | | |
| 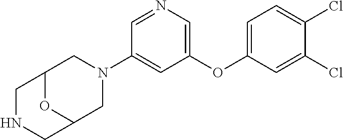 | 366 | 11 | 3.5 | 210 | 360 |
| 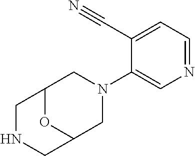 | 231 | | 860 | | |
| 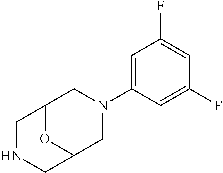 | 241 | 97 | 27 | | |
| 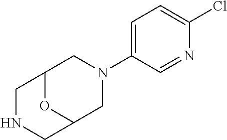 | 240 | 5.2 | 1.2 | 210 | 240 |
| 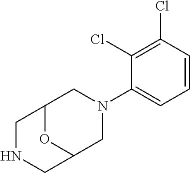 | 273 | | 9600 | | |
| 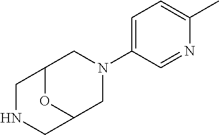 | 220 | 30 | 4.9 | | 600 |
| 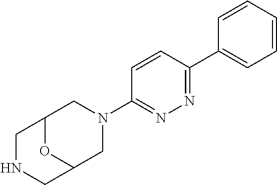 | 283 | | 380 | | |
| 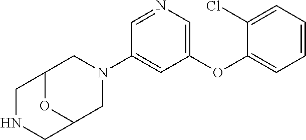 | 332 | 14 | 3.6 | | 430 |
| 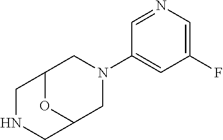 | 224 | 1.3 | 1.8 | 36 | 76 |
| 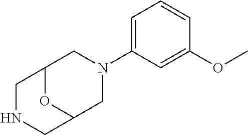 | 235 | 110 | 33 | | 800 |
| 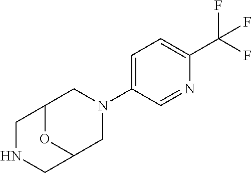 | 274 | 48 | 6.8 | | 1900 |
| 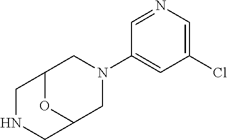 | 240 | 2.1 | 1.0 | 370 | 180 |
| 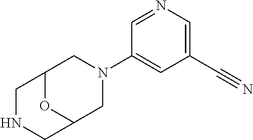 | 231 | 11 | 2.7 | | 2000 |
| 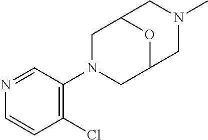 | 254 | | 180 | | |
| 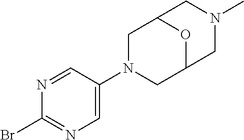 | 299 | | 2000 | | |
| 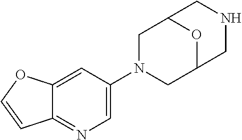 | 246 | 91 | 50 | | 5700 |
| 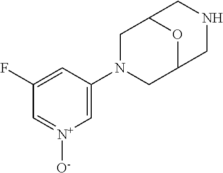 | 240 | | 590 | | 54000 |
| 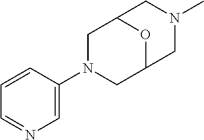 | 220 | 180 | 95 | | 5900 |
| 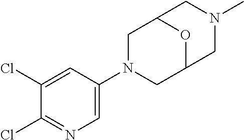 | 289 | 33 | 24 | | 4100 |
| 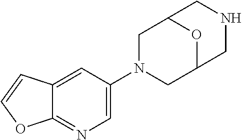 | 246 | 23 | 48 | | 6600 |
| 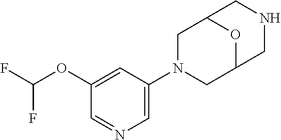 | 272 | 0.36 | 0.42 | | 1600 |
| 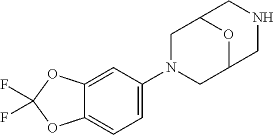 | 285 | | 700 | | 90000 |
| 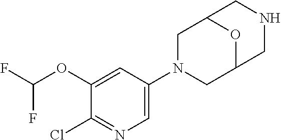 | 306 | 0.08 | 0.16 | | 560 |
| 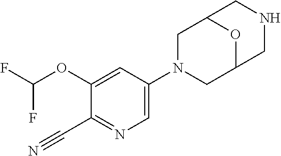 | 297 | 0.12 | 0.26 | | 940 |
| 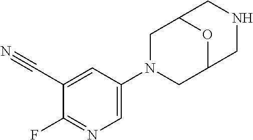 | 249 | 24 | 19 | | 16000 |
| 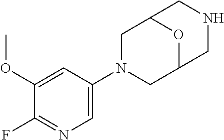 | 254 | 1.1 | 0.43 | | 1000 |
| 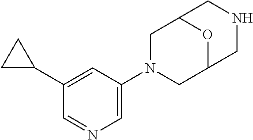 | 246 | 6.4 | 2.0 | | 1300 |
| 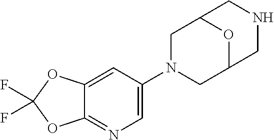 | 286 | 5.8 | 830 | | 9300 |
| 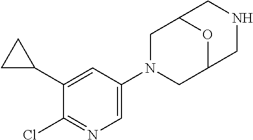 | 280 | 14 | 0.18 | | 87 |
| 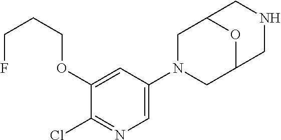 | 316 | 0.73 | 0.38 | | 1900 |
| 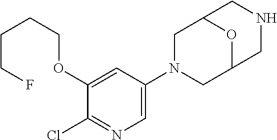 | 330 | 0.35 | 0.30 | | 1500 |
| 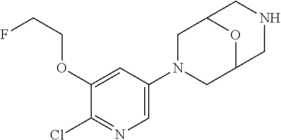 | 302 | 0.41 | 0.21 | | 1600 |

### Summary of Nicotinic Acetylcholine Receptor Data

Compounds of Table 1, representative of the present invention, exhibited inhibition constants (Ki values) at the rat and human α4β2 subtypes in the ranges of 0.1 nM to 1800 nM and 0.2 nM to 29,000 nM respectively, indicating high affinity for the α4β2 subtype. Ki values at the α7 subtype vary within the range of 14 nM to 61,000 nM, indicating lower affinity for the α7 subtype. Furthermore, some compounds failed to bind sufficiently in high through-put screening (HTS) to warrant Ki determination. This was more common for binding at the α7 subtype, as compared to the α4β2 subtype.

### Example 10

### Formalin Test

The formalin test in mice is a valid and reliable model of nociception and is sensitive for various classes of analgesic drugs. The noxious stimulus is an injection of dilute formalin (1% in saline) under the skin of the dorsal surface of the right hindpaw. The response is the amount of time the animals spend licking the injected paw. Two distinct periods of high licking activity can be identified, an early phase lasting the first 5 min and a late phase lasting from 20 to 30 min after the injection of formalin. See, Hunskaar et al., Pain, 1987, July; 30(1):103-14, incorporated by reference with regard to the test.

A formalin test was carried out in an open Plexiglas cage, with a mirror placed under the floor to allow an unobstructed view of the paws. Mice were allowed to acclimate for 15 min in the test cage before formalin injection. Each animal was injected with 20 μl of 2.5% formalin in the intraplantar region of the right hindpaw. Mice were then observed 0-5 min (Phase 1) and 20-45 min (Phase 2) post-formalin, and the amount of time spent (expressed in sec) licking the injected paw was recorded. Compound A or vehicle were injected s.c. 15 min before the formalin injection.

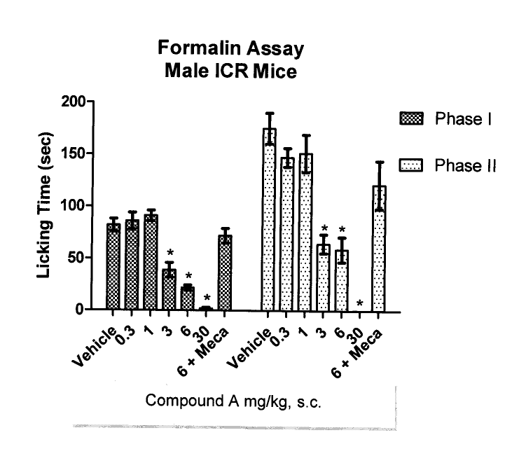

FIG. 1 illustrates the effects of Compound A in the formalin test (2.5%) in male ICR mice. As described, subject mice were pretreated with an s.c. injection of Compound A and 15 minutes later received formalin ipl. Compound A significantly reduced nociceptive behavior in both phases (F(1, 35)=41.8; P<0.0001, F(1,35)=24.8; P<0.0001, respectively) of the formalin test after s.c. administration. The lowest active dose was 3 mg/kg.

Additionally, the effects of Compound A were blocked by mecamylamine (2 mg/kg), thereby further supporting the activity through nAChR.

The specific pharmacological responses observed may vary according to and depending on the particular active compound selected or whether there are present pharmaceutical carriers, as well as the type of formulation and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

What is claimed is:

1. A compound of Formula I: 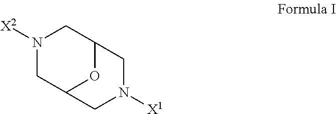
wherein:
X¹ is aryl (optionally substituted with one or more R groups) or heteroaryl (optionally substituted with one or more R groups);
each R independently is C₁₋₆ alkyl, C₂₋₆ alkenyl, C₂₋₆ alkynyl, C₃₋₈ cycloalkyl, —(CH₂)_{q}C₃₋₈ cycloalkyl, heterocyclyl, —(CH₂)_{q}heterocyclyl, aryl, —(CH₂)_{q}aryl, heteroaryl, —(CH₂)_{q}heteroaryl, halo, —OR^{I}, —NR^{I}R^{II}, C₁₋₆ haloalkyl , —CN, —NO₂, —C₂R^{I}, —SR^{I}, —N₃, —C(═O)NR^{I}R^{II}, —NR^{I}C(═O)R^{II}, —OC(═O)NR^{I}R^{II}, —NR^{I}C(═O)OR^{II}, —SO₂R^{I}, —SO₂NR^{I}R^{II}, or —NR^{I}SO₂R^{II};
each of R^{I} and R^{II} independently is hydrogen, C₁₋₆ alkyl, C₁₋₆ haloalkyl, C₃₋₈ cycloalkyl, —(CH₂)_{q}C₃₋₈ cycloalkyl, heterocyclyl, —(CH₂)_{q}heterocyclyl, aryl (optionally substituted with or more C₁₋₆ alkyl, halogen, or C₁₋₆ haloalkyl), —(CH₂)_{q}aryl (optionally substituted with one or more C₁₋₆ alkyl, halogen, or C₁₋₆ haloalkyl), heteroaryl (optionally substituted with one or more C₁₋₆ alkyl, halogen, or C₁₋₆ haloalkyl), or —(CH₂)_{q}heteroaryl (optionally substituted with one or more C₁₋₆ alkyl, halogen, or C₁₋₆ haloalkyl), or
R^{I} and R^{II} can combine together with the atoms to which they are attached to form a three to ten membered ring;
each q independently is 1, 2, 3, 4, 5, or 6;
X² is hydrogen, C₁₋₆ alkyl, cycloalkyl, —(CH₂)_{q}C₃₋₈cycloalkyl, —(CH₂)_{q}aryl, or —(CH₂)_{q}heteroaryl;
or a pharmaceutically acceptable salt thereof.

2. The compound of claim 1, wherein X¹ is unsubstituted or substituted pyridine, pyridazine, pyrimidine, phenyl, or pyrazine.

3. The compound of claim 2, wherein X¹ is substituted with one or more halogen, C₁₋₆ alkoxy, C₁₋₆ haloalkoxy, —(CH₂)_{q}C₃₋₈ cycloalkyl, C₁₋₆ alkyl, —CN, —OR^{I}, —NR^{I}R^{II}, or aryl.

4. The compound of claim 3, wherein X² is hydrogen or C₁₋₆ alkyl.

5. A compound selected from:
3-(6-chloropyridazin-3-yl)-9-oxa-3,7-diazabicyclo[3.3.1]nonane;
3-(5-methoxypyridin-3-yl)-9-oxa-3,7-diazabicyclo[3.3.1]nonane;
3-(5-isopropoxypyridin-3-yl)-9-oxa-3,7-diazabicyclo[3.3.1]nonane;
3-(5,6-dichloropyridin-3-yl)-9-oxa-3,7-diazabicyclo[3.3.1]nonane;
3-(5-trifluoromethylpyridin-3-yl)-9-oxa-3,7-diazabicyclo[3.3.1]nonane;
3-(5-methoxy-6-chloropyridin-3-yl)-9-oxa-3,7-diazabicyclo[3.3.1]nonane;
3-(5-(cyclopropylmethoxy)pyridin-3-yl)-9-oxa-3,7-diazabicyclo[3.3.1]nonane;
3-(5-bromopyridin-3-yl)-9-oxa-3,7-diazabicyclo[3.3.1]nonane;
3-(pyrimidin-5-yl)-9-oxa-3,7-diazabicyclo[3.3.1]nonane;
3-(pyridin-3-yl)-9-oxa-3,7-diazabicyclo[3.3.1]nonane;
3-(5-(3,4-dichlorophenoxy)pyridin-3-yl)-7-methyl-9-oxa-3,7-diazabicyclo[3.3.1]nonane;
3-(pyridin-4-yl)-7-methyl-9-oxa-3,7-diazabicyclo[3.3.1]nonane;
3-(6-trifluoromethyl-pyridin-3-yl)-7-methyl-9-oxa-3,7-diazabicyclo[3.3.1]nonane;
3-(5-fluoropyridin-3-yl)-7-methyl-9-oxa-3,7-diazabicyclo[3.3.1]nonane;
3-(6-fluoropyridin-3-yl)-7-methyl-9-oxa-3,7-diazabicyclo[3.3.1]nonane;
3-(2,3-difluorophenyl)-7-methyl-9-oxa-3,7-diazabicyclo[3.3.1]nonane;
3-(6-phenylpyridazin-3-yl)-7-methyl-9-oxa-3,7-diazabicyclo[3.3.1]nonane;
3-(4-cyanopyridin-3-yl)-7-methyl-9-oxa-3,7-diazabicyclo[3.3.1]nonane;
3-(pyrimidin-5-yl)-7-methyl-9-oxa-3,7-diazabicyclo[3.3.1]nonane;
3-(6-dimethylaminopyridin-3-yl)-7-methyl-9-oxa-3,7-diazabicyclo[3.3.1]nonane;
3-(3-methoxypyridin-2-yl)-7-methyl-9-oxa-3,7-diazabicyclo[3.3.1]nonane;
3-(5-methylpyridin-3-yl)-7-methyl-9-oxa-3,7-diazabicyclo[3.3.1]nonane;
3-(5-chloropyridin-3-yl)-7-methyl-9-oxa-3,7-diazabicyclo[3.3.1]nonane;
3-(3-methoxyphenyl)-7-methyl-9-oxa-3,7-diazabicyclo[3.3.1]nonane;
3-(3,5-difluorophenyl)-7-methyl-9-oxa-3,7-diazabicyclo[3.3.1]nonane;
3-(5-cyanopyridin-3-yl)-7-methyl-9-oxa-3,7-diazabicyclo[3.3.1]nonane;
3-(6-chloropyridazin-3-yl)-7-methyl-9-oxa-3,7-diazabicyclo[3.3.1]nonane;
3-(6-methoxypyridin-3-yl)-7-methyl-9-oxa-3,7-diazabicyclo[3.3.1]nonane;
3-(6-chloropyridin-3-yl)-7-methyl-9-oxa-3,7-diazabicyclo[3.3.1]nonane;
3-(5-(2-chlorophenoxy)pyridin-3-yl)-7-methyl-9-oxa-3,7-diazabicyclo[3.3.1]nonane;
3-(6-methoxypyridin-2-yl)-7-methyl-9-oxa-3,7-diazabicyclo[3.3.1]nonane;
3-(6-methylpyridin-3-yl)-7-methyl-9-oxa-3,7-diazabicyclo[3.3.1]nonane;
3-(6-(furan-3-yl)pyridazin-3-yl)-7-methyl-9-oxa-3,7-diazabicyclo[3.3.1]nonane;
3-(2,3-dichlorophenyl)-7-methyl-9-oxa-3,7-diazabicyclo[3.3.1]nonane;
3-(6-cyanopyridin-3-yl)-7-methyl-9-oxa-3,7-diazabicyclo[3.3.1]nonane;
3-(pyridin-2-yl)-7-methyl-9-oxa-3,7-diazabicyclo[3.3.1]nonane;
3-(3-methoxypyridin-2-yl)-9-oxa-3,7-diazabicyclo[3.3.1]nonane;
3-(2,3-difluorophenyl)-9-oxa-3,7-diazabicyclo[3.3.1]nonane;
3-(4-methoxypyridin-3-yl)-9-oxa-3,7-diazabicyclo[3.3.1]nonane;
3-(4-chloropyridin-3-yl)-9-oxa-3,7-diazabicyclo[3.3.1]nonane;
3-(6-fluoropyridin-3-yl)-9-oxa-3,7-diazabicyclo[3.3.1]nonane;
3-(6-cyanopyridin-3-yl)-9-oxa-3,7-diazabicyclo[3.3.1]nonane;
3-(5-methylpyridin-3-yl)-9-oxa-3,7-diazabicyclo[3.3.1]nonane;
3-(6-dimethylaminopyridin-3-yl)-9-oxa-3,7-diazabicyclo[3.3.1]nonane;
3-(pyrazin-2-yl)-9-oxa-3,7-diazabicyclo[3.3.1]nonane;
3-(pyridin-2-yl)-9-oxa-3,7-diazabicyclo[3.3.1]nonane;
3-(6-methoxypyridin-3-yl)-9-oxa-3,7-diazabicyclo[3.3.1]nonane;
3-(pyridin-4-yl)-9-oxa-3,7-diazabicyclo[3.3.1]nonane;
3-(5-(3,4-dichlorophenoxy)pyridin-3-yl)-9-oxa-3,7-diazabicyclo[3.3.1]nonane;
3-(4-cyanopyridin-3-yl)-9-oxa-3,7-diazabicyclo[3.3.1]nonane;
3-(3,5-difluorophenyl)-9-oxa-3,7-diazabicyclo[3.3.1]nonane;
3-(6-chloropyridin-3-yl)-9-oxa-3,7-diazabicyclo[3.3.1]nonane;
3-(6-methoxypyridin-2-yl)-9-oxa-3,7-diazabicyclo[3.3.1]nonane;
3-(2,3-dichlorophenyl)-9-oxa-3,7-diazabicyclo[3.3.1]nonane;
3-(6-methylpyridin-3-yl)-9-oxa-3,7-diazabicyclo[3.3.1]nonane;
3-(6-phenylpyridazin-3-yl)-9-oxa-3,7-diazabicyclo[3.3.1]nonane;
3-(5-(2-chlorophenoxy)pyridin-3-yl)-9-oxa-3,7-diazabicyclo[3.3.1]nonane;
3-(5-fluoropyridin-3-yl)-9-oxa-3,7-diazabicyclo[3.3.1]nonane;
3-(3-methoxyphenyl)-9-oxa-3,7-diazabicyclo[3.3.1]nonane;
3-(6-trifluoromethylpyridin-3-yl)-9-oxa-3,7-diazabicyclo[3.3.1]nonane;
3-(5-chloropyridin-3-yl)-9-oxa-3,7-diazabicyclo[3.3.1]nonane;
3-(2-phenylpyrimidin-5-yl)-9-oxa-3,7-diazabicyclo[3.3.1]nonane;
3-(6-(furan-3-yl)pyridazin-3-yl)-9-oxa-3,7-diazabicyclo[3.3.1]nonane;
3-(6-(pyridin-3-yl)pyridazin-3-yl)-9-oxa-3,7-diazabicyclo[3.3.1]nonane;
3-(5-cyanopyridin-3-yl)-9-oxa-3,7-diazabicyclo[3.3.1]nonane;
3-(pyrazin-2-yl)-7-methyl-9-oxa-3,7-diazabicyclo[3.3.1]nonane;
3-(2-phenylpyrimidin-5-yl)-7-methyl-9-oxa-3,7-diazabicyclo[3.3.1]nonane;
3-(6-(pyridin-3-yl)pyridazin-3-yl)-7-methyl-9-oxa-3,7-diazabicyclo[3.3.1]nonane;
3-(4-chloropyridin-3-yl)-7-methyl-9-oxa-3,7-diazabicyclo[3.3.1]nonane;
3-(2-bromopyrimidin-5-yl)-9-oxa-3,7-diazabicyclo[3.3.1]nonane;
3-(4-methoxypyridin-3-yl)-7-methyl-9-oxa-3,7-diazabicyclo[3.3.1]nonane; or
3-(2-bromopyrimidin-5-yl)-7-methyl-9-oxa-3,7-diazabicyclo[3.3.1]nonane;
3-(furo[3,2-b]pyridin-6-yl)-9-oxa-3,7-diazabicyclo[3.3.1]nonane;
3-(5-fluoro-pyridine-1-oxide-3-yl)-9-oxa-3,7-diazabicyclo[3.3.1]nonane;
3-methyl-7-(pyridin-3-yl)-9-oxa-3,7-diazabicyclo[3.3.1]nonane;
3-methyl-7-(5,6-dichloropyridin-3-yl)-9-oxa-3,7-diazabicyclo[3.3.1]nonane;
3-(furo[2,3-b]pyridin-5-yl)-9-oxa-3,7-diazabicyclo[3.3.1]nonane;
3-(5-(difluoromethoxy)pyridin-3-yl)-9-oxa-3,7-diazabicyclo[3.3.1]nonane;
3-(2,2-difluorobenzo[d][1,3]dioxol-5-yl)-9-oxa-3,7-diazabicyclo[3.3.1]nonane;
3-(6-chloro-5-(difluoromethoxy)pyridin-3-yl)-9-oxa-3,7-diazabicyclo[3.3.1]nonane;
3-(6-cyano-5-(difluoromethoxy)pyridin-3-yl)-9-oxa-3,7-diazabicyclo[3.3.1]nonane;
3-(5-cyano-6-fluoropyridin-3-yl)-9-oxa-3,7-diazabicyclo[3.3.1]nonane;
3-(5-methoxy-6-fluoropyridin-3-yl)-9-oxa-3,7-diazabicyclo[3.3.1]nonane;
3-(5-cyclopropylpyridin-3-yl)-9-oxa-3,7-diazabicyclo[3.3.1]nonane;
3-(2,2-difluoro-[1,3]dioxolo[4,5-b]pyridine-5-yl)-9-oxa-3,7-diazabicyclo[3.3.1]nonane;
3-(5-cyclopropyl-6-chloropyridin-3-yl)-9-oxa-3,7-diazabicyclo[3.3.1]nonane;
3-(5-(3-fluoropropoxy)-6-chloropyridin-3-yl)-9-oxa-3,7-diazabicyclo[3.3.1]nonane;
3-(5-(4-fluorobutoxy)-6-chloropyridin-3-yl)-9-oxa-3,7-diazabicyclo[3.3.1]nonane; and
3-(5-(2-fluoroethoxy)-6-chloropyridin-3-yl)-9-oxa-3,7-diazabicyclo[3.3.1]nonane;
or a pharmaceutically acceptable salt thereof.

6. A compound: 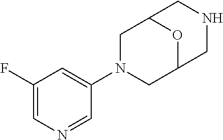
or a pharmaceutically acceptable salt thereof.

7. A pharmaceutical composition comprising a compound as claimed in claim 1 and a pharmaceutically acceptable carrier.

8. A method for the treatment or prevention of a disease or condition mediated by a neuronal nicotinic receptor comprising the administration of a compound as claimed in claim 1.

9. The method of claim 8, wherein the neuronal nicotinic receptor is of the α4β2 or α7 subtype.

10. The method of claim 9, wherein the condition is pain.

11. The method of claim 10, wherein the pain is neuropathic pain.

12. A method for the treatment or prevention of a disease or condition mediated by a neuronal nicotinic receptor comprising the administration of a compound as claimed in claim 6.

13. The method of claim 12, wherein the neuronal nicotinic receptor is of the α4β2 or α7 subtype

14. The method of claim 13, wherein the condition is pain.

15. The method of claim 14, wherein the pain is neuropathic pain.

16. (canceled)

17. (canceled)
